SEQUENCE LISTING (141 sequences)

SEQ ID NO: 1 - protein, 55 aa, Artificial sequence
  Relaxin 800800
DSWMEEVIKLCGRELVRAQIAICGMSTWSKRQLYSALANKCCHVGCTKRSLARFC

SEQ ID NO: 2 - protein, 59 aa, Artificial sequence
  Relaxin 800801
DSWMEEVIKLCGRELVRAQIAICGMSTWSKRSLKRQLYSALANKCCHVGCTKRSLARFC

SEQ ID NO: 3 - protein, 66 aa, Artificial sequence
  Relaxin 800802
DSWMEEVIKLCGRELVRAQIAICGMSTWSKRKPTGYGSRKKRQLYSALANKCCHVGCTKRSLARFC

SEQ ID NO: 4 - protein, 66 aa, Artificial sequence
  Relaxin 800802-1
DSWMEEVIKLCGRDLVRAQIAICGMSTWSKRKPTGYGSRKKRDLYSALANKCCHVGCTKRSLARFC

SEQ ID NO: 5 - protein, 65 aa, Artificial sequence
  Relaxin 800803
DSWMEEVIKLCGRELVRAQIAICGMSTWSKRKPTGYGSRKRQLYSALANKCCHVGCTKRSLARFC

SEQ ID NO: 6 - protein, 65 aa, Artificial sequence
  Relaxin 800803-1
DSWMEEVIKLCGRDLVRAQIAICGMSTWSKRKPTGYGSRKRDLYSALANKCCHVGCTKRSLARFC

SEQ ID NO: 7 - protein, 61 aa, Artificial sequence
  Relaxin 800805
DSWMEEVIKLCGRELVRAQIAICGMSTWSKRGGGPRRQLYSALANKCCHVGCTKRSLARFC

SEQ ID NO: 8 - protein, 61 aa, Artificial sequence
  Relaxin 800805-1
DSWMEEVIKLCGRDLVRAQIAICGMSTWSKRGGGPRRDLYSALANKCCHVGCTKRSLARFC

SEQ ID NO: 9 - protein, 61 aa, Artificial sequence
  Relaxin 800806
DSWMEEVIKLCGRELVRAQIAICGMSTWSKRGGGPKRQLYSALANKCCHVGCTKRSLARFC

SEQ ID NO: 10 - protein, 61 aa, Artificial sequence
  Relaxin 800806-1
DSWMEEVIKLCGRDLVRAQIAICGMSTWSKRGGGPKRDLYSALANKCCHVGCTKRSLARFC

SEQ ID NO: 11 - protein, 61 aa, Artificial sequence
  Relaxin 800808
EEGEPKDSWMEEVIKLCGRELVRAQIAICGMSTWSKRQLYSALANKCCHVGCTKRSLARFC

SEQ ID NO: 12 - protein, 61 aa, Artificial sequence
  Relaxin 800808-1
EEGEPKDSWMEEVIKLCGRDLVRAQIAICGMSTWSKRDLYSALANKCCHVGCTKRSLARFC

SEQ ID NO: 13 - protein, 62 aa, Artificial sequence
  Relaxin 800809
EEGEPKRDSWMEEVIKLCGRELVRAQIAICGMSTWSKRQLYSALANKCCHVGCTKRSLARFC

SEQ ID NO: 14 - protein, 62 aa, Artificial sequence
  Relaxin 800809-1
EEGEPKRDSWMEEVIKLCGRDLVRAQIAICGMSTWSKRDLYSALANKCCHVGCTKRSLARFC

SEQ ID NO: 15 - protein, 55 aa, Artificial sequence
  Relaxin 800810
DSWMEEVIKLCGRDLVRAQIAICGMSTWSKRQLYSALANKCCHVGCTKRSLARFC

SEQ ID NO: 16 - protein, 55 aa, Artificial sequence
  Relaxin 800810-1
DSWMEEVIKLCGRDLVRAQIAICGMSTWSKRDLYSALANKCCHVGCTKRSLARFC

SEQ ID NO: 17 - protein, 64 aa, Artificial sequence
  Relaxin 800811
DSWMEEVIKLCGRDLVRAQIAICGMSTWSKRKPTGYGSKRQLYSALANKCCHVGCTKRSLARFC

SEQ ID NO: 18 - protein, 64 aa, Artificial sequence
  Relaxin 800813
DSWMEEVIKLCGRDLVRAQIAICGMSTWSKRKPTGYGSKRELYSALANKCCHVGCTKRSLARFC

SEQ ID NO: 19 - protein, 64 aa, Artificial sequence
  Relaxin 800814
DSWMEEVIKLCGRDLVRAQIAICGMSTWSKRKPTGYGSKRDLYSALANKCCHVGCTKRSLARFC

SEQ ID NO: 20 - protein, 64 aa, Artificial sequence
  Relaxin 800816
DSWMEEVIKLCGRNLVRAQIAICGMSTWSKRKPTGYGSKRQLYSALANKCCHVGCTKRSLARFC

SEQ ID NO: 21 - protein, 64 aa, Artificial sequence
  Relaxin 800847Y
DSWMEEVIKLCGRELVRAQIAICGMSTWSKRKPTGYGSKRDLYSALANKCCHVGCTKRSLARFC

SEQ ID NO: 22 - protein, 62 aa, Artificial sequence
  Relaxin 800851Y
WMEEVIKLCGRDLVRAQIAICGMSTWSKRKPTGYGSKRDLYSALANKCCHVGCTKRSLARFC

SEQ ID NO: 23 - protein, 77 aa, Artificial sequence
  Relaxin 800828
MKKNIAFLLKRDSWMEEVIKLCGRELVRAQIAICGMSTWSKRKPTGYGSRKKRQLYSALANKCCHVGCTKRSLARFC

SEQ ID NO: 24 - protein, 77 aa, Artificial sequence
  Relaxin 800843
MKKNIAFLLKRDSWMEEVIKLCGRELVRAQIAICGMSTWSKRKPTGYGSRKKRWLYSALANKCCHVGCTKRSLARFC

SEQ ID NO: 25 - protein, 75 aa, Artificial sequence
  Relaxin 800847
MKKNIAFLLKRDSWMEEVIKLCGRELVRAQIAICGMSTWSKRKPTGYGSKRDLYSALANKCCHVGCTKRSLARFC

SEQ ID NO: 26 - protein, 73 aa, Artificial sequence
  Relaxin 800851
MKKNIAFLLKRWMEEVIKLCGRDLVRAQIAICGMSTWSKRKPTGYGSKRDLYSALANKCCHVGCTKRSLARFC

SEQ ID NO: 27 - protein, 13 aa, Artificial sequence
  L2
KRKPTGYGSRKKR

SEQ ID NO: 28 - protein, 12 aa, Artificial sequence
  L3
KRKPTGYGSRKR

SEQ ID NO: 29 - protein, 8 aa, Artificial sequence
  L4
KRGGGPRR

SEQ ID NO: 30 - protein, 8 aa, Artificial sequence
  L5
KRGGGPKR

SEQ ID NO: 31 - protein, 11 aa, Artificial sequence
  L6
KRKPTGYGSKR

SEQ ID NO: 32 - protein, 6 aa, Artificial sequence
  L7
KRSLKR

SEQ ID NO: 33 - protein, 6 aa, Artificial sequence
  S1
EEGEPK

SEQ ID NO: 34 - protein, 7 aa, Artificial sequence
  S2
EEGEPKR

SEQ ID NO: 35 - protein, 11 aa, Artificial sequence
  S3
MKKNIAFLLKR

SEQ ID NO: 36 - DNA, 34 bp, Artificial sequence
  800-Primer 1
ctcgaggattcttggatggaagaagttattaagt

SEQ ID NO: 37 - DNA, 59 bp, Artificial sequence
  800-Primer 2
caatttgagctctaaccaattctctaccacacaacttaataacttcttccatccaagaa

SEQ ID NO: 38 - DNA, 59 bp, Artificial sequence
  800-Primer 3
agagaattggttagagctcaaattgctatttgtggtatgtctacttggtctaagagaca

SEQ ID NO: 39 - DNA, 59 bp, Artificial sequence
  800-Primer 4
atgacaacacttgttagccaaagcagagtacaattgtctcttagaccaagtagacatac

SEQ ID NO: 40 - DNA, 59 bp, Artificial sequence
  800-Primer 5
ctttggctaacaagtgttgtcatgttggttgtactaagagatctttggctagattttgt

SEQ ID NO: 41 - DNA, 34 bp, Artificial sequence
  800-Primer 6
gaattcttaacaaaatctagccaaagatctctta

SEQ ID NO: 42 - DNA, 168 bp, Artificial sequence
  800800 precursor DNA sequence
gattcttggatggaagaagttattaagttgtgtggtagagaattggttagagctcaaattgctatttgtggtatgtctacttggtctaagagacaattgtactctgctttggctaacaagtgttgtcatgttggttgtactaagagatctttggctagattttgttaa

SEQ ID NO: 43 - DNA, 46 bp, Artificial sequence
  801-Primer 1
gtatgtctacttggtctaagagatctttgaagagacaattgtactc

SEQ ID NO: 44 - DNA, 46 bp, Artificial sequence
  801-Primer 2
gagtacaattgtctcttcaaagatctcttagaccaagtagacatac

SEQ ID NO: 45 - DNA, 180 bp, Artificial sequence
  800801 precursor DNA sequence
gattcttggatggaagaagttattaagttgtgtggtagagaattggttagagctcaaattgctatttgtggtatgtctacttggtctaagagatctttgaagagacaattgtactctgctttggctaacaagtgttgtcatgttggttgtactaagagatctttggctagattttgttaa

SEQ ID NO: 46 - DNA, 66 bp, Artificial sequence
  802-Primer 1
gtctacttggtctaagagaaagccaactggttacggttctagaaagaagagacaattgtactctgc

SEQ ID NO: 47 - DNA, 66 bp, Artificial sequence
  802-Primer 2
gcagagtacaattgtctcttctttctagaaccgtaaccagttggctttctcttagaccaagtagac

SEQ ID NO: 48 - DNA, 201 bp, Artificial sequence
  800802 precursor DNA sequence
gattcttggatggaagaagttattaagttgtgtggtagagaattggttagagctcaaattgctatttgtggtatgtctacttggtctaagagaaagccaactggttacggttctagaaagaagagacaattgtactctgctttggctaacaagtgttgtcatgttggttgtactaagagatctttggctagattttgttaa

SEQ ID NO: 49 - DNA, 43 bp, Artificial sequence
  802-1-Primer 1
gttattaagttgtgtggtagagatttggttagagctcaaattg

SEQ ID NO: 50 - DNA, 43 bp, Artificial sequence
  802-1-Primer 2
caatttgagctctaaccaaatctctaccacacaacttaataac

SEQ ID NO: 51 - DNA, 36 bp, Artificial sequence
  802-1-Primer 3
gttctagaaagaagagagatttgtactctgctttgg

SEQ ID NO: 52 - DNA, 36 bp, Artificial sequence
  802-1-Primer 4
ccaaagcagagtacaaatctctcttctttctagaac

SEQ ID NO: 53 - DNA, 201 bp, Artificial sequence
  800802-1 precursor DNA sequence
gattcttggatggaagaagttattaagttgtgtggtagagatttggttagagctcaaattgctatttgtggtatgtctacttggtctaagagaaagccaactggttacggttctagaaagaagagagatttgtactctgctttggctaacaagtgttgtcatgttggttgtactaagagatctttggctagattttgttaa

SEQ ID NO: 54 - DNA, 63 bp, Artificial sequence
  803-Primer 1
gtctacttggtctaagagaaagccaactggttacggttctagaaagagacaattgtactctgc

SEQ ID NO: 55 - DNA, 63 bp, Artificial sequence
  803-Primer 2
gcagagtacaattgtctctttctagaaccgtaaccagttggctttctcttagaccaagtagac

SEQ ID NO: 56 - DNA, 198 bp, Artificial sequence
  800803 precursor DNA sequence
gattcttggatggaagaagttattaagttgtgtggtagagaattggttagagctcaaattgctatttgtggtatgtctacttggtctaagagaaagccaactggttacggttctagaaagagacaattgtactctgctttggctaacaagtgttgtcatgttggttgtactaagagatctttggctagattttgttaa

SEQ ID NO: 57 - DNA, 43 bp, Artificial sequence; identical to SEQ ID NO: 49
  803-1-Primer 1

SEQ ID NO: 58 - DNA, 43 bp, Artificial sequence; identical to SEQ ID NO: 50
  803-1-Primer 2

SEQ ID NO: 59 - DNA, 35 bp, Artificial sequence
  803-1-Primer 3
cggttctagaaagagagatttgtactctgctttgg

SEQ ID NO: 60 - DNA, 35 bp, Artificial sequence
  803-1-Primer 4
ccaaagcagagtacaaatctctctttctagaaccg

SEQ ID NO: 61 - DNA, 198 bp, Artificial sequence
  800803-1 precursor DNA sequence
gattcttggatggaagaagttattaagttgtgtggtagagatttggttagagctcaaattgctatttgtggtatgtctacttggtctaagagaaagccaactggttacggttctagaaagagagatttgtactctgctttggctaacaagtgttgtcatgttggttgtactaagagatctttggctagattttgttaa

SEQ ID NO: 62 - DNA, 55 bp, Artificial sequence
  805-Primer 1
gtctacttggtctaagagaggtggtggtccaagaagacaattgtactctgctttg

SEQ ID NO: 63 - DNA, 55 bp, Artificial sequence
  805- Primer 2
caaagcagagtacaattgtcttcttggaccaccacctctcttagaccaagtagac

SEQ ID NO: 64 - DNA, 186 bp, Artificial sequence
  800805 precursor DNA sequence
gattcttggatggaagaagttattaagttgtgtggtagagaattggttagagctcaaattgctatttgtggtatgtctacttggtctaagagaggtggtggtccaagaagacaattgtactctgctttggctaacaagtgttgtcatgttggttgtactaagagatctttggctagattttgttaa

SEQ ID NO: 65 - DNA, 43 bp, Artificial sequence; identical to SEQ ID NO: 49
  805-1-Primer 1

SEQ ID NO: 66 - DNA, 43 bp, Artificial sequence; identical to SEQ ID NO: 50
  805-1-Primer 2

SEQ ID NO: 67 - DNA, 34 bp, Artificial sequence
  805-1-Primer 3
ggtggtccaagaagagatttgtactctgctttgg

SEQ ID NO: 68 - DNA, 34 bp, Artificial sequence
  805-1-Primer 4
ccaaagcagagtacaaatctcttcttggaccacc

SEQ ID NO: 69 - DNA, 186 bp, Artificial sequence
  800805-1 precursor DNA sequence
gattcttggatggaagaagttattaagttgtgtggtagagatttggttagagctcaaattgctatttgtggtatgtctacttggtctaagagaggtggtggtccaagaagagatttgtactctgctttggctaacaagtgttgtcatgttggttgtactaagagatctttggctagattttgttaa

SEQ ID NO: 70 - DNA, 52 bp, Artificial sequence
  806-Primer 1
gtctacttggtctaagagaggtggtggtccaaagagacaattgtactctgct

SEQ ID NO: 71 - DNA, 52 bp, Artificial sequence
  806-Primer 2
agcagagtacaattgtctctttggaccaccacctctcttagaccaagtagac

SEQ ID NO: 72 - DNA, 186 bp, Artificial sequence
  800806 precursor DNA sequence
gattcttggatggaagaagttattaagttgtgtggtagagaattggttagagctcaaattgctatttgtggtatgtctacttggtctaagagaggtggtggtccaaagagacaattgtactctgctttggctaacaagtgttgtcatgttggttgtactaagagatctttggctagattttgttaa

SEQ ID NO: 73 - DNA, 43 bp, Artificial sequence; identical to SEQ ID NO: 49
  806-1-Primer 1

SEQ ID NO: 74 - DNA, 43 bp, Artificial sequence; identical to SEQ ID NO: 50
  806-1-Primer 2

SEQ ID NO: 75 - DNA, 37 bp, Artificial sequence
  806-1-Primer 3
ggtggtggtccaaagagagatttgtactctgctttgg

SEQ ID NO: 76 - DNA, 37 bp, Artificial sequence
  806-1-Primer 4
ccaaagcagagtacaaatctctctttggaccaccacc

SEQ ID NO: 77 - DNA, 186 bp, Artificial sequence
  800806-1 precursor DNA sequence
gattcttggatggaagaagttattaagttgtgtggtagagatttggttagagctcaaattgctatttgtggtatgtctacttggtctaagagaggtggtggtccaaagagagatttgtactctgctttggctaacaagtgttgtcatgttggttgtactaagagatctttggctagattttgttaa

SEQ ID NO: 78 - DNA, 57 bp, Artificial sequence
  808-Primer 1
gggtatctctcgagaaaagagaagaaggtgaaccaaaggattcttggatggaagaag

SEQ ID NO: 79 - DNA, 57 bp, Artificial sequence
  808-Primer 2
cttcttccatccaagaatcctttggttcaccttcttctcttttctcgagagataccc

SEQ ID NO: 80 - DNA, 186 bp, Artificial sequence
  800808 precursor DNA sequence
gaagaaggtgaaccaaaggattcttggatggaagaagttattaagttgtgtggtagagaattggttagagctcaaattgctatttgtggtatgtctacttggtctaagagacaattgtactctgctttggctaacaagtgttgtcatgttggttgtactaagagatctttggctagattttgttaa

SEQ ID NO: 81 - DNA, 43 bp, Artificial sequence; identical to SEQ ID NO: 49
  808-1-Primer 1

SEQ ID NO: 82 - DNA, 43 bp, Artificial sequence; identical to SEQ ID NO: 50
  808-1-Primer 2

SEQ ID NO: 83 - DNA, 36 bp, Artificial sequence
  808-1-Primer 3
ctacttggtctaagagagatttgtactctgctttgg

SEQ ID NO: 84 - DNA, 36 bp, Artificial sequence
  808-1-Primer 4
ccaaagcagagtacaaatctctcttagaccaagtag

SEQ ID NO: 85 - DNA, 186 bp, Artificial sequence
  808-1 precursor DNA sequence
gaagaaggtgaaccaaaggattcttggatggaagaagttattaagttgtgtggtagagatttggttagagctcaaattgctatttgtggtatgtctacttggtctaagagagatttgtactctgctttggctaacaagtgttgtcatgttggttgtactaagagatctttggctagattttgttaa

SEQ ID NO: 86 - DNA, 60 bp, Artificial sequence
  809-Primer 1
gggtatctctcgagaaaagagaagaaggtgaaccaaagagagattcttggatggaagaag

SEQ ID NO: 87 - DNA, 60 bp, Artificial sequence
  809-Primer 2
cttcttccatccaagaatctctctttggttcaccttcttctcttttctcgagagataccc

SEQ ID NO: 88 - DNA, 189 bp, Artificial sequence
  800809 precursor DNA sequence
gaagaaggtgaaccaaagagagattcttggatggaagaagttattaagttgtgtggtagagaattggttagagctcaaattgctatttgtggtatgtctacttggtctaagagacaattgtactctgctttggctaacaagtgttgtcatgttggttgtactaagagatctttggctagattttgttaa

SEQ ID NO: 89 - DNA, 43 bp, Artificial sequence; identical to SEQ ID NO: 49
  809-1-Primer 1

SEQ ID NO: 90 - DNA, 43 bp, Artificial sequence; identical to SEQ ID NO: 50
  809-1-Primer 2

SEQ ID NO: 91 - DNA, 36 bp, Artificial sequence; identical to SEQ ID NO: 83
  809-1-Primer 3

SEQ ID NO: 92 - DNA, 36 bp, Artificial sequence; identical to SEQ ID NO: 84
  809-1-Primer 4

SEQ ID NO: 93 - DNA, 189 bp, Artificial sequence
  800809-1 precursor DNA sequence
gaagaaggtgaaccaaagagagattcttggatggaagaagttattaagttgtgtggtagagatttggttagagctcaaattgctatttgtggtatgtctacttggtctaagagagatttgtactctgctttggctaacaagtgttgtcatgttggttgtactaagagatctttggctagattttgttaa

SEQ ID NO: 94 - DNA, 43 bp, Artificial sequence; identical to SEQ ID NO: 49
  810-Primer 1

SEQ ID NO: 95 - DNA, 43 bp, Artificial sequence; identical to SEQ ID NO: 50
  810-Primer 2

SEQ ID NO: 96 - DNA, 168 bp, Artificial sequence
  800810 precursor DNA sequence
gattcttggatggaagaagttattaagttgtgtggtagagatttggttagagctcaaattgctatttgtggtatgtctacttggtctaagagacaattgtactctgctttggctaacaagtgttgtcatgttggttgtactaagagatctttggctagattttgttaa

SEQ ID NO: 97 - DNA, 36 bp, Artificial sequence; identical to SEQ ID NO: 83
  810-1-Primer 1

SEQ ID NO: 98 - DNA, 36 bp, Artificial sequence; identical to SEQ ID NO: 84
  810-1-Primer 2

SEQ ID NO: 99 - DNA, 168 bp, Artificial sequence
  800810-1 precursor DNA sequence
gattcttggatggaagaagttattaagttgtgtggtagagatttggttagagctcaaattgctatttgtggtatgtctacttggtctaagagagatttgtactctgctttggctaacaagtgttgtcatgttggttgtactaagagatctttggctagattttgttaa

SEQ ID NO: 100 - DNA, 43 bp, Artificial sequence; identical to SEQ ID NO: 49
  811-Primer 1

SEQ ID NO: 101 - DNA, 43 bp, Artificial sequence; identical to SEQ ID NO: 50
  811-Primer 2

SEQ ID NO: 102 - DNA, 195 bp, Artificial sequence
  800811 precursor DNA sequence
gattcttggatggaagaagttattaagttgtgtggtagagatttggttagagctcaaattgctatttgtggtatgtctacttggtctaagagaaagccaactggttacggttctaagagacaattgtactctgctttggctaacaagtgttgtcatgttggttgtactaagagatctttggctagattttgttaa

SEQ ID NO: 103 - DNA, 40 bp, Artificial sequence
  813-Primer 1
ctggttacggttctaagagagaattgtactctgctttggc

SEQ ID NO: 104 - DNA, 40 bp, Artificial sequence
  813-Primer 2
gccaaagcagagtacaattctctcttagaaccgtaaccag

SEQ ID NO: 105 - DNA, 195 bp, Artificial sequence
  800813 precursor DNA sequence
gattcttggatggaagaagttattaagttgtgtggtagagatttggttagagctcaaattgctatttgtggtatgtctacttggtctaagagaaagccaactggttacggttctaagagagaattgtactctgctttggctaacaagtgttgtcatgttggttgtactaagagatctttggctagattttgttaa

SEQ ID NO: 106 - DNA, 40 bp, Artificial sequence
  814-Primer 1
ctggttacggttctaagagagatttgtactctgctttggc

SEQ ID NO: 107 - DNA, 40 bp, Artificial sequence
  814-Primer 2
gccaaagcagagtacaaatctctcttagaaccgtaaccag

SEQ ID NO: 108 - DNA, 195 bp, Artificial sequence
  800814 precursor DNA sequence
gattcttggatggaagaagttattaagttgtgtggtagagatttggttagagctcaaattgctatttgtggtatgtctacttggtctaagagaaagccaactggttacggttctaagagagatttgtactctgctttggctaacaagtgttgtcatgttggttgtactaagagatctttggctagattttgttaa

SEQ ID NO: 109 - DNA, 41 bp, Artificial sequence
  816-Primer 1
attaagttgtgtggtagaaacttggttagagctcaaattgc

SEQ ID NO: 110 - DNA, 41 bp, Artificial sequence
  816-Primer 2
gcaatttgagctctaaccaagtttctaccacacaacttaat

SEQ ID NO: 111 - DNA, 195 bp, Artificial sequence
  800816 precursor DNA sequence
gattcttggatggaagaagttattaagttgtgtggtagaaacttggttagagctcaaattgctatttgtggtatgtctacttggtctaagagaaagccaactggttacggttctaagagacaattgtactctgctttggctaacaagtgttgtcatgttggttgtactaagagatctttggctagattttgttaa

SEQ ID NO: 112 - DNA, 34 bp, Artificial sequence
  847Y-Primer 1
aagttgtgtggtagagaattggttagagctcaaa

SEQ ID NO: 113 - DNA, 34 bp, Artificial sequence
  847Y-Primer 2
tttgagctctaaccaattctctaccacacaactt

SEQ ID NO: 114 - DNA, 195 bp, Artificial sequence
  800847Y precursor DNA sequence
gattcttggatggaagaagttattaagttgtgtggtagagaattggttagagctcaaattgctatttgtggtatgtctacttggtctaagagaaagccaactggttacggttctaagagagatttgtactctgctttggctaacaagtgttgtcatgttggttgtactaagagatctttggctagattttgttaa

SEQ ID NO: 115 - DNA, 42 bp, Artificial sequence
  851Y-Primer 1
ggggtatctctcgagaaaagatggatggaagaagttattaag

SEQ ID NO: 116 - DNA, 42 bp, Artificial sequence
  851Y-Primer 2
cttaataacttcttccatccatcttttctcgagagatacccc

SEQ ID NO: 117 - DNA, 189 bp, Artificial sequence
  800851Y precursor DNA sequence
tggatggaagaagttattaagttgtgtggtagagatttggttagagctcaaattgctatttgtggtatgtctacttggtctaagagaaagccaactggttacggttctaagagagatttgtactctgctttggctaacaagtgttgtcatgttggttgtactaagagatctttggctagattttgttaa

SEQ ID NO: 118 - DNA, 50 bp, Artificial sequence
  828-Primer 1
catatgaagaaaaacatcgcgttcctgctgaaacgtgactcttggatgga

SEQ ID NO: 119 - DNA, 59 bp, Artificial sequence
  828-Primer 2
gcacgaaccagttcacgaccgcacagtttgataacttcttccatccaagagtcacgttt

SEQ ID NO: 120 - DNA, 59 bp, Artificial sequence
  828-Primer 3
cgtgaactggttcgtgcgcaaattgcgatctgcggtatgtctacctggtctaaacgtaa

SEQ ID NO: 121 - DNA, 59 bp, Artificial sequence
  828-Primer4
cagctgacgttttttacgagaaccgtaaccggtcggtttacgtttagaccaggtagaca

SEQ ID NO: 122 - DNA, 59 bp, Artificial sequence
  828-Primer 5
ctcgtaaaaaacgtcagctgtactctgcgctggcgaacaaatgctgccacgttggttgc

SEQ ID NO: 123 - DNA, 52 bp, Artificial sequence
  828-Primer 6
ggatccttagcagaaacgcgccagagaacgtttggtgcaaccaacgtggcag

SEQ ID NO: 124 - DNA, 243 bp, Artificial sequence
  800828 precursor DNA sequence
catatgaagaaaaacatcgcgttcctgctgaaacgtgactcttggatggaagaagttatcaaactgtgcggtcgtgaactggttcgtgcgcaaattgcgatctgcggtatgtctacctggtctaaacgtaaaccgaccggttacggttctcgtaaaaaacgtcagctgtactctgcgctggcgaacaaatgctgccacgttggttgcaccaaacgttctctggcgcgtttctgctaaggatcc

SEQ ID NO: 125 - DNA, 35 bp, Artificial sequence
  843-Primer 1
gttctcgtaaaaaacgttggctgtactctgcgctg

SEQ ID NO: 126 - DNA, 35 bp, Artificial sequence
  843-Primer 2
cagcgcagagtacagccaacgttttttacgagaac

SEQ ID NO: 127 - DNA, 234 bp, Artificial sequence
  800843 precursor DNA sequence
atgaagaaaaacatcgcgttcctgctgaaacgtgactcttggatggaagaagttatcaaactgtgcggtcgtgaactggttcgtgcgcaaattgcgatctgcggtatgtctacctggtctaaacgtaaaccgaccggttacggttctcgtaaaaaacgttggctgtactctgcgctggcgaacaaatgctgccacgttggttgcaccaaacgttctctggcgcgtttctgctaa

SEQ ID NO: 128 - DNA, 35 bp, Artificial sequence
  847-Primer 1
caaactgtgcggtcgtgaactggttcgtgcgcaaa

SEQ ID NO: 129 - DNA, 35 bp, Artificial sequence
  847-Primer 2
tttgcgcacgaaccagttcacgaccgcacagtttg

SEQ ID NO: 130 - DNA, 228 bp, Artificial sequence
  800847 precursor DNA sequence
atgaagaaaaacatcgcgttcctgctgaaacgtgactcttggatggaagaagttatcaaactgtgcggtcgtgaactggttcgtgcgcaaattgcgatctgcggtatgtctacctggtctaaacgtaaaccgaccggttacggttctaaacgtgacctgtactctgcgctggcgaacaaatgctgccacgttggttgcaccaaacgttctctggcgcgtttctgctaa

SEQ ID NO: 131 - DNA, 37 bp, Artificial sequence
  851-Primer 1
cgcgttcctgctgaaacgttggatggaagaagttatc

SEQ ID NO: 132 - DNA, 37 bp, Artificial sequence
  851-Primer 2
gataacttcttccatccaacgtttcagcaggaacgcg

SEQ ID NO: 133 - DNA, 222 bp, Artificial sequence
  800851 precursor DNA sequence
atgaagaaaaacatcgcgttcctgctgaaacgttggatggaagaagttatcaaactgtgcggtcgtgacctggttcgtgcgcaaattgcgatctgcggtatgtctacctggtctaaacgtaaaccgaccggttacggttctaaacgtgacctgtactctgcgctggcgaacaaatgctgccacgttggttgcaccaaacgttctctggcgcgtttctgctaa

SEQ ID NO: 134 - protein, 29 aa, Artificial sequence
  human relaxin chain B
DSWMEEVIKLCGRELVRAQIAICGMSTWS

SEQ ID NO: 135 - protein, 24 aa, Artificial sequence
  human relaxin chain A
QLYSALANKCCHVGCTKRSLARFC

SEQ ID NO: 136 - protein, 24 aa, Artificial sequence
  human relaxin analogue 800813 - chain A EA1
ELYSALANKCCHVGCTKRSLARFC

SEQ ID NO: 137 - protein, 29 aa, Artificial sequence
  human relaxin analogue chain B DB14
DSWMEEVIKLCGRDLVRAQIAICGMSTWS

SEQ ID NO: 138 - protein, 24 aa, Artificial sequence
  human relaxin analogue chain A DA1
DLYSALANKCCHVGCTKRSLARFC

SEQ ID NO: 139 - protein, 29 aa, Artificial sequence
  human relaxin analogue 800816 - chain B NB14
DSWMEEVIKLCGRNLVRAQIAICGMSTWS

SEQ ID NO: 140 - protein, 27 aa, Artificial sequence
  human relaxin analogue 800851 - chain B DelB1B2,DB1
WMEEVIKLCGRDLVRAQIAICGMSTWS

SEQ ID NO: 141 - protein, 24 aa, Artificial sequence
  human relaxin analogue 800843 - chain AWA1
WLYSALANKCCHVGCTKRSLARFC

(19) United States
(12) Patent Application Publication
(10) Pub. No.: US 2016/0326230 A1
(43) Pub. Date: Nov. 10, 2016

(54) HUMAN RELAXIN ANALOGUE, PHARMACEUTICAL COMPOSITION OF SAME, AND PHARMACEUTICAL APPLICATION OF SAME

(71) Applicants: SHANGHAI HENGRUI PHARMACEUTICAL CO., LTD., Shanghai (CN); JIANGSU HENGRUI MEDICINE CO., LTD., Lianyungang, Jiangsu (CN)

(72) Inventors: Lianshan ZHANG, Lianyungang, Jiangsu (CN); Jiajian LIU, Shanghai (CN); Guoqing CAO, Shanghai (CN)

(21) Appl. No.: 15/033,421

(22) PCT Filed: Oct. 10, 2014
(86) PCT No.: PCT/CN2014/088280

§ 371 Date: Apr. 29, 2016

(30) Foreign Application Priority Data
Nov. 7, 2013 (CN) 201310548221.8

(51) Int. Cl. C07K 14/64; A61K 38/22; A61K 9/00; A61K 47/48

(52) U.S. Cl. CPC C07K 14/64; A61K 47/48215; A61K 38/2221; A61K 9/0019

(57) ABSTRACT

Disclosed are a human relaxin analogue, a polynucleotide encoding the human relaxin analogue, a pharmaceutical composition comprising the human relaxin analogue, and a pharmaceutical application of the human relaxin analogue. Also disclosed is a derivative of the human relaxin analogue.

### CROSS REFERENCE TO RELATED APPLICATION

This application is a Section 371 of International Application No. PCT/CN2014/088280, filed Oct. 10, 2014, which was published in the Chinese language on May 14, 2015, under International Publication No. WO 2015/067113 A1, and the disclosure of which is incorporated herein by reference.

### REFERENCE TO SEQUENCE LISTING SUBMITTED ELECTRONICALLY

This application contains a sequence listing, which is submitted electronically via EFS-Web as an ASCII formatted sequence listing with a file name “Sequence_Listing.TXT”, creation date of Apr. 29, 2016, and having a size of 44.2 kilobytes. The sequence listing submitted via EFS-Web is part of the specification and is herein incorporated by reference in its entirety.

### FIELD OF THE INVENTION

The present disclosure relates to a novel human relaxin analog and to pharmaceutical compositions and uses thereof.

### BACKGROUND OF THE INVENTION

Relaxin (referred to as RLX) is a polypeptide hormone secreted by the mammalian corpus luteum. Relaxin has a variety of physiological functions in vivo, including stretching the pubic ligament, inhibiting uterine contractions, softening the cervix, stimulating breast development, and affecting galactosis. In 1926, relaxin was first discovered by Frederick Hisaw while studying pelvic girdle changes during pregnancy. Relaxin was considered a double-chain protein during 1970s and 1980s. The structure of relaxin is similar to that of insulin. It has been verified that relaxin is a member of the family of peptide hormones. The Homo sapeins relaxin protein family is encoded by seven genes: RLN1, RLN2, RLN3 (also referred to as INSL7), INSL3/RLF, INSL4/EPIL, INSL5/RIF2 and INSL6/RIF1. Most of the relaxins in human circulation are encoded by the RLN2 gene. The translation product of RLN2 is a relaxin precursor, comprising (from N-terminus to C-terminus): a signal peptide that is 24 amino acid residues long, a B chain that is 29 amino acid residues long, a linker peptide that is 104 to 107 amino acid residues long, and an A chain that is about 24 amino acid residues long.

It is clear that relaxin not only plays an important role in pregnancy, but it also has an effect on the structure and function of vessels in non-pregnant animals. Relaxin has a wide range of biological effects, including maintaining homeostasis of the internal environment during pregnancy and aging in mammals, anti-inflammation, cardio-protection, dilation of blood vessels, promotion of wound healing, and particularly anti-fibrotic effects. Heart and kidney diseases are caused by various factors that ultimately lead to fibrosis, structural changes and loss of function. Therefore, the effective inhibition of fibrosis is important in maintaining organ function. Thus, the anti-fibrotic effect of relaxin may be a potentially effective future anti-fibrotic therapy. More importantly, relaxin is produced by the heart, and it protects the heart and modulates extracellular matrix via local receptors. Relaxin has been successfully used to ameliorate cardiac fibrosis in various animal models. Thus, relaxin is expected to be useful in the treatment of human heart fibrotic diseases (Xiaojun Du, Juan Zhou, Baker Heart Institute).

In the prior art, the human relaxin (hRelaxin, wild-type) precursor was expressed in E. coli, refolded after purification, digested by carboxypeptidase B (CPB) and trypsin respectively in a two-step reaction, and then purified to obtain the active product, relaxin. Deficiencies in the prior art include protein refolding, two-step digestion and re-purification steps are required in the production process. The multiple steps required greatly reduces yield and increases cost, since each step involves a loss of protein. Furthermore, exogenous enzymes (e.g., CPB, trypsin, etc.) are costly, and the quality of the enzymes affects the quality and yield of the final product.

Another drawback is that the expressed wild-type relaxin exhibits low activity, resulting in an increased amount of recombinant protein needed for treatment, and thus an increased cost. Furthermore, the low activity of the expressed wild-type relaxin makes it difficult to further develop protein modifications, since such modification would damage the protein activity.

To address the above-mentioned deficiencies of the prior art, the present invention provides a series of novel molecules by altering amino acid(s) of wild-type relaxin at specific site(s), and provides novel human relaxin analogs that can efficiently solve the above-mentioned problems. The disclosed technical solution possesses the following advantages:

1. Amino acid changes at particular sites of relaxin help the analogs to be correctly folded in yeast cells, and the c-peptides of chain A and chain B are removed by the endogenous enzyme system of the yeast cells in the process. Complete active molecules with functional physiological properties are obtained and secreted extracellularly. The active molecules are obtained by one-step purification. As a result, the downstream production steps, such as refolding, digestion and purification that were required in the prior art, can be omitted.

2. Amino acid changes at particular sites of relaxin make the structure of relaxin analogs more suitable for intracellular folding, digestion and secretion. Furthermore, the biological activity of relaxin is improved. Thus, the amount of recombinant protein required in a dosage unit can be reduced, leading to lower costs. Meanwhile, the development of molecular modifications would be much easier, due to the increased activity of such analogs.

Human relaxin analogs with specific sequences are obtained in the present invention via designs (e.g., replacement or deletion of specific amino acid(s) in relaxin sequence, etc.). Protein folding and enzyme digestion of the relaxin analog precursor proteins can be carried out in eukaryotic expression systems (cells), and when they are secreted into the fermentation broth, the human relaxin analogs of the present disclosure are mature, intact and functional. Moreover, the biological activity of the relaxin analogs disclosed herein is at least two times higher than that of wild-type.

### SUMMARY OF THE INVENTION

The present disclosure provides a human relaxin analog, comprising chain A and chain B, wherein the amino acid sequences of the chain A and chain B are represented respectively by the following formulas:

chain A: A₁LYSALANKCCHVGCTKRSLARFC (24 amino acid residues),

chain B: DSWMEEVIKLCGRB₁₄LVRAQIAICGMSTWS (29 amino acid residues),

wherein A₁ is selected from the group consisting of Q, D, E, and W;

wherein B₁₄ is selected from the group consisting of E, D and N;

wherein the residues at B₁ and B₂ are optionally absent simultaneously;

wherein when A₁ is Q, B₁₄ is neither E nor D.

In one embodiment of the present disclosure, a human relaxin analog as described above is provided, comprising chain A and chain B, wherein the amino acid sequences of the chain A and chain B are represented by the following formulas, respectively:

chain A: A₁LYSALANKCCHVGCTKRSLARFC (24 amino acid residues),

chain B: DSWMEEVIKLCGRB₁₄LVRAQIAICGMSTWS (29 amino acid residues);

wherein A₁ is selected from the group consisting of Q, D, E, and W;

wherein B₁₄ is selected from the group consisting of E, D and N;

wherein when A₁ is Q, B₁₄ is neither E nor D.

In one embodiment of the present disclosure, A₁ is D, E or W, preferably D.

In one embodiment of the present disclosure, A₁ is D, and B₁₄ is D. In a particular example, a human relaxin analog comprises SEQ ID NO: 134 (chain A) and SEQ ID NO: 135 (chain B), and the chain A and chain B are linked to each other by a disulfide bond.

In one embodiment of the present disclosure, B₁₄ is D.

In one embodiment of the present disclosure, the human relaxin analog as described above comprises the amino acid sequences of chain A and chain B selected from the group consisting of:

SEQ ID NO: 137

Chain B: DSWMEEVIKLCGRDLVRAQIAICGMSTWS

SEQ ID NO: 138

chain A: DLYSALANKCCHVGCTKRSLARFC;

SEQ ID NO: 137

chain B: DSWMEEVIKLCGRDLVRAQIAICGMSTWS

SEQ ID NO: 135

chain A: QLYSALANKCCHVGCTKRSLARFC;

SEQ ID NO: 137

chain B: DSWMEEVIKLCGRDLVRAQIAICGMSTWS

SEQ ID NO: 136

chain A: ELYSALANKCCHVGCTKRSLARFC;

SEQ ID NO: 139

chain B: DSWMEEVIKLCGRNLVRAQIAICGMSTWS

SEQ ID NO: 135

chain A: QLYSALANKCCHVGCTKRSLARFC;

SEQ ID NO: 140

chain B: WMEEVIKLCGRDLVRAQIAICGMSTWS

SEQ ID NO: 138

chain A: DLYSALANKCCHVGCTKRSLARFC;

SEQ ID NO: 134

chain B: DSWMEEVIKLCGRELVRAQIAICGMSTWS

SEQ ID NO: 141

chain A: WLYSALANKCCHVGCTKRSLARFC;
and

SEQ ID NO: 134

chain B: DSWMEEVIKLCGRELVRAQIAICGMSTWS

SEQ ID NO: 138

chain A: DLYSALANKCCHVGCTKRSLARFC.

In one embodiment of the present disclosure, the human relaxin analog defined above comprises chain B linked to chain A by a linker sequence (referred to L in this disclosure), wherein the linker sequence is 1 to 15 amino acid residues long, preferably 2 to 8 amino acid residues long, and the amino acid sequence of the linker sequence is selected from the group consisting of:

L1: KR,

SEQ ID NO: 27

L2: KRKPTGYGSRKKR,,

SEQ ID NO: 28

L3: KRKPTGYGSRKR,,

SEQ ID NO: 29

L4: KRGGGPRR,,

SEQ ID NO: 30

L5: KRGGGPKR,,

SEQ ID NO: 31

L6: KRKPTGYGSKR,,
and

SEQ ID NO: 32

L7: KRSLKR.

In one embodiment of the present disclosure, provided is the human relaxin analog described above, wherein the N terminus of the human relaxin analog is linked to a signal peptide sequence (referred to S in this disclosure), wherein the signal peptide sequence is 4 to 15 amino acid residues long, preferably 6 to 11 amino acid residues long, and the amino acid sequence of the signal peptide sequence is selected from the group consisting of:

SEQ ID NO: 33

S1: EEGEPK,,

SEQ ID NO: 34

S2: EEGEPKR,,
and

SEQ ID NO: 35

S3: MKKNIAFLLKR.

In one embodiment of the present disclosure, provided is an expression precursor, which is used for preparing the human relaxin analog described above, and the amino acid sequence of the expression precursor is one or more sequences selected from the group consisting of SEQ ID NOs: 1-26.

TABLE 1
Summary of Expression precursors of human relaxin analogs

| | Amino acid sequence of | |
|---|---|---|
| | human relaxin analog expression | |
| Example | precursor (SEQ ID NO) | abbreviation |
| relaxin | DSWMEEVIKLCGRELVRAQIAICGMSTW | B(wt)-L1-A(wt) |
| 800800 | SKRQLYSALANKCCHVGCTKRSLARFC | |
| | (SEQ ID NO: 1) | |
| relaxin | DSWMEEVIKLCGRELVRAQIAICGMSTW | B(wt)-L7-A(wt) |
| 800801 | SKRSLKRQLYSALANKCCHVGCTKRSLA | |
| | RFC | |
| | (SEQ ID NO: 2) | |
| relaxin | DSWMEEVIKLCGRELVRAQIAICGMSTW | B(wt)-L2-A(wt) |
| 800802 | SKRKPTGYGSRKKRQLYSALANKCCHV | |
| | GCTKRSLARFC | |
| | (SEQ ID NO: 3) | |
| relaxin | DSWMEEVIKLCGRDLVRAQIAICGMSTW | D^{B14}-L2-D^{A1} |
| 800802-1 | SKRKPTGYGSRKKRDLYSALANKCCHV | |
| | GCTKRSLARFC | |
| | (SEQ ID NO: 4) | |
| relaxin | DSWMEEVIKLCGRELVRAQIAICGMSTW | B(wt)-L3-A(wt) |
| 800803 | SKRKPTGYGSRKRQLYSALANKCCHVG | |
| | CTKRSLARFC | |
| | (SEQ ID NO: 5) | |
| relaxin | DSWMEEVIKLCGRDLVRAQIAICGMSTW | D^{B14}-L3-D^{A1} |
| 800803-1 | SKRKPTGYGSRKRDLYSALANKCCHVG | |
| | CTKRSLARFC | |
| | (SEQ ID NO: 6) | |
| relaxin | DSWMEEVIKLCGRELVRAQIAICGMSTW | B(wt)-L4-A(wt) |
| 800805 | SKRGGGPRRQLYSALANKCCHVGCTKRS | |
| | LARFC | |
| | (SEQ ID NO: 7) | |
| relaxin | DSWMEEVIKLCGRDLVRAQIAICGMSTW | D^{B14}-L4-D^{A1} |
| 800805-1 | SKRGGGPRRDLYSALANKCCHVGCTKRS | |
| | LARFC | |
| | (SEQ ID NO: 8) | |
| relaxin | DSWMEEVIKLCGRELVRAQIAICGMSTW | B(wt)-L5-A(wt) |
| 800806 | SKRGGGPKRQLYSALANKCCHVGCTKR | |
| | SLARFC | |
| | (SEQ ID NO: 9) | |
| relaxin | DSWMEEVIKLCGRDLVRAQIAICGMSTW | D^{B14}-L5-D^{A1} |
| 800806-1 | SKRGGGPKRDLYSALANKCCHVGCTKR | |
| | SLARFC | |
| | (SEQ ID NO: 10) | |
| relaxin | EEGEPKDSWMEEVIKLCGRELVRAQIAIC | S1-B(wt)-L1-A(wt) |
| 800808 | GMSTWSKRQLYSALANKCCHVGCTKRS | |
| | LARFC | |
| | (SEQ ID NO: 11) | |
| relaxin | EEGEPKDSWMEEVIKLCGRDLVRAQIAIC | S1-D^{B14}-L1-D^{A1} |
| 800808-1 | GMSTWSKRDLYSALANKCCHVGCTKRS | |
| | LARFC | |
| | (SEQ ID NO: 12) | |
| relaxin | EEGEPKRDSWMEEVIKLCGRELVRAQIAI | S2-B(wt)-L1-A(wt) |
| 800809 | CGMSTWSKRQLYSALANKCCHVGCTKR | |
| | SLARFC | |
| | (SEQ ID NO: 13) | |
| relaxin | EEGEPKRDSWMEEVIKLCGRDLVRAQIAI | S2-D^{B14}-L1-D^{A1} |
| 800809-1 | CGMSTWSKRDLYSALANKCCHVGCTKR | |
| | SLARFC | |
| | (SEQ ID NO: 14) | |
| relaxin | DSWMEEVIKLCGRDLVRAQIAICGMSTW | D^{B14}-L1-A(wt) |
| 800810 | SKRQLYSALANKCCHVGCTKRSLARFC | |
| | (SEQ ID NO: 15) | |
| relaxin | DSWMEEVIKLCGRDLVRAQIAICGMSTW | D^{B14}-L1-D^{A1} |
| 800810-1 | SKRDLYSALANKCCHVGCTKRSLARFC | |
| | (SEQ ID NO: 16) | |
| relaxin | DSWMEEVIKLCGRDLVRAQIAICGMSTW | D^{B14}-L6-A(wt) |
| 800811 | SKRKPTGYGSKRQLYSALANKCCHVGCT | |
| | KRSLARFC | |
| | (SEQ ID NO: 17) | |
| relaxin | DSWMEEVIKLCGRDLVRAQIAICGMSTW | D^{B14}-L6-E^{A1} |
| 800813 | SKRKPTGYGSKRELYSALANKCCHVGCT | |
| | KRSLARFC | |
| | (SEQ ID NO: 18) | |
| relaxin | DSWMEEVIKLCGRDLVRAQIAICGMSTW | D^{B14}-L6-D^{A1} |
| 800814 | SKRKPTGYGSKRDLYSALANKCCHVGCT | |
| | KRSLARFC | |
| | (SEQ ID NO: 19) | |
| relaxin | DSWMEEVIKLCGRNLVRAQIAICGMSTW | N^{B14}-L6-A(wt) |
| 800816 | SKRKPTGYGSKRQLYSALANKCCHVGCT | |
| | KRSLARFC | |
| | (SEQ ID NO: 20) | |
| relaxin | DSWMEEVIKLCGRELVRAQIAICGMSTW | B(wt)-L6-D^{A1} |
| 800847Y | SKRKPTGYGSKRDLYSALANKCCHVGCT | |
| | KRSLARFC | |
| | (SEQ ID NO: 21) | |
| relaxin | WMEEVIKLCGRDLVRAQIAICGMSTWSK | D^{B14}-L6-D^{A1} |
| 800851Y | RKPTGYGSKRDLYSALANKCCHVGCTK | |
| | RSLARFC | |
| | (SEQ ID NO: 22) | |
| relaxin | MKKNIAFLLKRDSWMEEVIKLCGRELVR | S3-B(wt)-L2-A(wt) |
| 800828 | AQIAICGMSTWSKRKPTGYGSRKKRQLY | |
| | SALANKCCHVGCTKRSLARFC | |
| | (SEQ ID NO: 23) | |
| relaxin | MKKNIAFLLKRDSWMEEVIKLCGRELVR | S3-B(wt)-L2-W^{A1} |
| 800843 | AQIAICGMSTWSKRKPTGYGSRKKRWLY | |
| | SALANKCCHVGCTKRSLARFC | |
| | (SEQ ID NO: 24) | |
| relaxin | MKKNIAFLLKRDSWMEEVIKLCGRELVR | S3-B(wt)-L6-D^{A1} |
| 800847 | AQIAICGMSTWSKRKPTGYGSKRDLYSA | |
| | LANKCCHVGCTKRSLARFC | |
| | (SEQ ID NO: 25) | |
| relaxin | MKKNIAFLLKRWMEEVIKLCGRDLVRA | S3-Del^{B1,B2}-D^{B14}-L6- |
| 800851 | QIAICGMSTWSKRKPTGYGSKRDLYSAL | D^{A1} |
| | ANKCCHVGCTKRSLARFC | |
| | (SEQ ID NO: 26) | |

The present disclosure further provides human relaxin analog derivatives obtained by a PEG-modification of the human relaxin analogs described above. In other words, the human relaxin analogs described above are modified by a polyethylene glycol (PEG) molecule. In some embodiments, the molecular weight of the PEG is 5 to 100 KDa; in some embodiments, 10 to 80 KDa; in some embodiments, 15 to 45 KDa; in some embodiments, 20 to 40 KDa. In some embodiments, the PEG molecule is a branched-chain type or a linear-chain type.

The present disclosure further provides a polynucleotide encoding an expression precursor of a human relaxin analog described above. The polynucleotide is selected from DNA or RNA. It will be appreciated by those skilled in the art that the complementary sequence of the polynucleotide is also within the scope of this disclosure.

The present disclosure further provides an expression vector comprising the polynucleotide as described above.

The present disclosure further provides a host cell transformed with the expression vector as described above. In some embodiments, the host cell is a bacterial cell. In some particular embodiments, the host cell is an E. coli cell. In other embodiments, the host cell is a yeast cell. In some particular embodiments, the host cell is a Pichia pastoris cell.

The present disclosure further provides a pharmaceutical composition which comprises or consists of the following components:

a) one or more human relaxin analog(s) as described above, and/or one or more human relaxin analog derivative(s) as described above, and

b) one or more pharmaceutically acceptable carrier(s), diluent(s) or excipient(s).

The present disclosure further provides an injectable solution of a human relaxin analog or derivative thereof, wherein the injectable solution contains a dissolved form or dissoluble form of the pharmaceutical composition as described above. It should be understood that the dry powder or lyophilized powder form of the injectable solution is also encompassed within the scope of this disclosure.

The present disclosure further provides the use of a human relaxin analog as described above, a human relaxin analog derivative as described above, the pharmaceutical composition as described above, or the injectable solution described above, in the preparation of a medicament for the treatment or prevention of fibrotic disease or cardiovascular disease, for reference, see, e.g., Cardiovascular effects of relaxin; from basic science to clinical therapy, Nat. Rev. Cardiol. 7, 48-58 (2010); Relaxin decreases renal interstitial fibrosis and slows progression of renal disease, Kidney International, Vol. 59(2001), pp. 876-882.

The present disclosure further provides a method for treating or preventing fibrotic disease or cardiovascular disease, the method comprising a step of administering to a subject in need thereof a therapeutically effective amount of a human relaxin analog as described above or a derivative thereof, or of the pharmaceutical composition as described above.

### DETAILED DESCRIPTION OF THE INVENTION

### Terms

For a better understanding of the present disclosure, certain technical and scientific terms are specifically defined below. Unless specifically defined elsewhere in this document, all other technical and scientific terms used herein have the meaning commonly understood by one of ordinary skill in the art to which this disclosure belongs.

As used herein, the single-letter code and the three-letter code for amino acids are as described in J. Biol. Chem, 243, (1968) p 3558.

“Administered”, “administration”, “treatment” and “treating”, as they apply to an animal, human, experimental subject, cell, tissue, organ, or biological fluid, refer to contact of an exogenous pharmaceutical, therapeutic, diagnostic agent, or composition to the animal, human, subject, cell, tissue, organ, or biological fluid. “Administered”, “administration”, “treatment” and “treating” refer, e.g., to therapeutic, pharmacokinetic, diagnostic, research, and experimental methods. Treatment of a cell encompasses contact of a reagent with the cell, as well as contact of a reagent with a fluid, wherein the fluid is in contact with the cell. “Administered”, “administration”, “treatment” and “treating” also mean in vitro and ex vivo treatments of a cell, by a reagent, diagnostic, binding composition, or by another cell. “Treatment” as it applies to a human, veterinary, or research subject, refers to therapeutic treatment, prophylactic or preventative measures, or to research and diagnostic applications.

“Treat” or “treating” means to internally or externally administer a therapeutic agent, such as a composition containing any of the linked compounds of the present disclosure, to a patient with one or more disease symptoms for which the agent has a known therapeutic activity. Typically, the therapeutic agent is administered in an amount effective to alleviate one or more disease symptoms in the treated patient or population, regardless of how the effective amount works, either by inducing the regression of such symptom(s) or by preventing such symptom(s) from developing into a clinically measurable amount. The amount of a therapeutic agent that is effective to alleviate any particular disease symptom (also referred to as the “therapeutically effective amount”) can vary according to factors such as the disease state, the age and weight of the patient, as well as the ability of the drug to elicit a desired response in the patient. Whether a disease symptom has been alleviated can be assessed by any clinical measurement typically used by physicians or other skilled healthcare providers to assess the severity or progression status of that symptom. Though the embodiments of the present disclosure (e.g., a treatment method or manufactured product) can vary in alleviating the target disease symptom(s) among patients, the embodiments should alleviate the target disease symptom(s) in patients with statistical significance, as determined by any statistical test known in the art, such as the Student's t-test, the chi square test, the U-test according to Mann and Whitney, the Kruskal-Wallis test (H-test), the Jonckheere-Terpstra-test and the Wilcoxon-test.

Unless stated clearly elsewhere in this document, “relaxin” or “human relaxin” as used herein is human relaxin RLN2, which comprises a full length sequence or a partial sequence having biological activity. Human relaxin contains chain A and chain B which are linked together via disulfide bond(s). The sequence description of human relaxin used herein refers to GenBank accession number: EAW58770. Relaxin 800828 is wild-type and used as a positive control in this disclosure.

From the amino-terminus to the carboxy-terminus, the human relaxin analog precursor according to the present disclosure comprises: a signal peptide sequence, a chain B or mutant thereof, a linker, and a chain A or mutant thereof. The length of the signal peptide sequence is 4 to 15 amino acid residues, preferably 6 to 11 amino acid residues. The length of the chain B or mutant thereof is about 29 amino acid residues. The length of the linker sequence is 1 to 15 amino acid residues, preferably 2 to 8 amino acid residues. The length of the chain A or variant thereof is about 24 amino acid residues.

“Mutant” as used herein refers to amino acid modifications, replacements, substitutions, or deletions in a sequence. Preferable mutations in chain B include the replacement of the E at the 14^{th} amino acid residue (B₁₄ for short) with D. Preferable mutations in chain A include the replacement of the Q at the 1^{st} amino acid residue (referred to as A1) with D.

Sequence abbreviations used herein are as follows: Ln represents linker sequences (e.g. L1 to L6) in this disclosure, Sn represents signal peptide sequences (e.g., S1, S2, S3) of this disclosure; D^{A1} indicates that the mutation at the 1^{st} amino acid of chain A is D, D^{B14} indicates that the mutation at the 14^{th} amino acid of chain B is D, Del^{B1,B2} indicates the absence of the 1^{st} and 2^{nd} amino acids of chain B; A(wt) represents wild type chain A without any mutations, and B(wt) represents wild type chain B without any mutations.

“Conservative modification” or “conservative substitution” refers to a substitution of an amino acid in a protein by another amino acid residue having similar characteristics (e.g. charge, side-chain size, hydrophobicity/hydrophilicity, backbone conformation and rigidity, etc.), such that the change can frequently be made without altering the biological activity of the protein. Those of skill in this art recognize that, in general, single amino acid substitutions in non-essential regions of a polypeptide do not substantially alter their biological activity (see, e.g., Watson et al. (1987) Molecular Biology of the Gene, The Benjamin/Cummings Pub. Co., p. 224, 4th Ed.). In addition, substitutions of structurally or functionally similar amino acid residues are less likely to disrupt biological activity.

“Effective amount” encompasses an amount sufficient to ameliorate or prevent a symptom or sign of the medical condition. Effective amount also means an amount sufficient to allow or facilitate diagnosis. An effective amount for a particular subject can vary depending on factors such as the condition being treated, the overall health of the patient or veterinary subject, the administration method, the route and dose of administration, and the severity of side effects. An effective amount can be the maximal dose or dosing protocol that avoids significant side effects or toxic effects.

“Exogenous” refers to substances that are produced outside of an organism, cell, or human body. “Endogenous” refers to substances that are produced within a cell, organism, or human body.

As used herein, the terms “cell”, “cell line” and “cell culture” are used interchangeably, and all such designations include their progeny. Thus, the words “transformant” and “transformed cell” include the primary subject cell as well as cultures derived therefrom, regardless of the passage number. It is also understood that all progeny may not be precisely identical in DNA content, due to deliberate or inadvertent mutations. Mutant progeny that have the same function or biological activity as that of originally transformed cell are included. Where distinct designations are intended, it will be clear from the context.

As used herein, “polymerase chain reaction” or “PCR” refers to an amplification procedure or technique as described in, e.g., U.S. Pat. No. 4,683,195. Generally, sequence information from the ends of the region of interest or beyond must be known, such that oligonucleotide primers can be designed. These primers will be identical or similar in sequence to opposite strands of the template to be amplified. The 5′ terminal nucleotides of the two primers can coincide with the ends of the amplified material. PCR can be used to amplify specific RNA sequences, specific DNA sequences from total genomic DNA, cDNA transcribed from total cellular RNA, bacteriophage or plasmid sequences, etc. See generally Mullis et al. (1987) Cold Spring Harbor Symp. Quant. Biol. 51:263; Erlich, ed., (1989) PCR TECHNOLOGY (Stockton Press, N.Y.). As used herein, PCR is considered to be one, but not the only, example of a nucleic acid polymerase reaction method for amplifying a nucleic acid test sample. Such method comprises the use of a known nucleic acid as a primer and a nucleic acid polymerase to amplify or generate a specific portion of the nucleic acid.

“Optional” or “optionally” means that the event or situation that follows can, but does not necessarily, occur.

“Pharmaceutical composition” refers to a mixture comprising one or more analogs or precursors according to the present disclosure, and additional chemical components, wherein said additional components are physiologically or pharmaceutically acceptable carriers and excipients. Said additional components serve to promote the administration to an organism, facilitating the absorption of the active ingredient and thereby producing a biological effect.

The transformation procedure of a host cell referred to in this disclosure is well known to those of skill in the art. The obtained transformant can be cultured by a conventional method, and it can express a polypeptide that is encoded by a gene of the present disclosure. The culture medium used herein is selected from various conventional culture mediums depending on the host cell used. The host cells are cultured under a suitable condition. Human relaxin analogs can be released from the expression precursor protein using chemical and/or enzymatic methods well known to those skilled in the art, such as trypsin, carboxypeptidase B, or lysine endopeptidase C digestion, etc.

### EXAMPLES

### Example 1

### Cloning and Expression of Recombinant Human Relaxin 800800

1. Construction of Recombinant Human Relaxin 800800 Expression Vector

The DNA sequence of codon-optimized human relaxin 800800 was synthesized by an Overlapping PCR method. Six single-stranded DNA fragments that were synthesized by Invitrogen were used as synthetic primers. Their sequences were as follows:

800-primer 1

SEQ ID NO: 36

CTCGAGGATTCTTGGATGGAAGAAGTTATTAAGT
800-primer 2

SEQ ID NO: 37

CAATTTGAGCTCTAACCAATTCTCTACCACACAACTTAATAACTTCTTC
CATCCAAGAA
800-primer 3

SEQ ID NO: 38

AGAGAATTGGTTAGAGCTCAAATTGCTATTTGTGGTATGTCTACTTGGT
CTAAGAGACA
800-primer 4

SEQ ID NO: 39

ATGACAACACTTGTTAGCCAAAGCAGAGTACAATTGTCTCTTAGACCA
AGTAGACATAC
800-primer 5

SEQ ID NO: 40

CTTTGGCTAACAAGTGTTGTCATGTTGGTTGTACTAAGAGATCTTTGGC
TAGATTTTGT
800-primer 6

SEQ ID NO: 41

GAATTCTTAACAAAATCTAGCCAAAGATCTCTTA

Relaxin was synthesized using a KOD plus PCR kit (TOYOBO, Cat. KOD-201), and the reaction was performed by two-step PCR.

The conditions of PCR step 1 were as follows—

25 μL reaction volume: 2.5 μL of 10×KOD buffer, 2.5 μL of 2 mM dNTPs, 1 μL each of primers 1, 2, 3, 4, 5, 6 (10 μM), 0.5 μL of KOD plus, 1 μL of 25 mM MgSO₄, 12.5 μL of ddH₂O.

Thermocycling program: one cycle of 94° C. for 5 minutes; 30 amplification cycles of 94° C. for 30 seconds, 60° C. for 30 seconds, and 68° C. for 30 seconds; then one cycle of 68° C. for 30 minutes to terminate the PCR amplification.

The conditions of PCR step 2 were as follows—

25 μL reaction volume: 2.5 μL, of 10×KOD buffer, 2.5 μL of 2 mM dNTPs, 1 μL each of primers 1 and 6 (10 μM), 1 μL of PCR step 1 product, 0.5 μL of KOD plus, 1 μL of 25 mM MgSO4, 15.5 μL of ddH₂O.

Thermocycling program: one cycle of 94° C. for 5 minutes; 30 amplification cycles of 94° C. for 30 seconds, and 68° C. for 60 seconds; one cycle of 68° C. for 10 minutes to terminate PCR amplification.

PCR-generated DNA sequences and a pPIC9K expression vector (Invitrogen, Cat. K1750-01) were digested using EcoRI and Xho I, respectively (New England Biolabs, Cat. R0101S/R0146V). The resulting fragments of interest were recovered by 1.2% agarose gel electrophoresis, ligated using T4 DNA ligase (New England Biolabs, Cat. M0202V), and transformed into DH5a competent cells (Tiangen, Cat. CB101-02). Positive clones were picked, and sequenced by Invitrogen. The DNA sequence of 800800 precursor is as follows:

SEQ ID NO: 42

GATTCTTGGATGGAAGAAGTTATTAAGTTGTGTGGTAGAGAATTGGTTA
GAGCTCAAATTGCTATTTGTGGTATGTCTACTTGGTCTAAGAGACAATT
GTACTCTGCTTTGGCTAACAAGTGTTGTCATGTTGGTTGTACTAAGAGA
TCTTTGGCTAGATTTTGTTAA

The amino acid sequence coded by the DNA sequence above is as follows:

SEQ ID NO: 1

DSWMEEVIKLCGRELVRAQIAICGMSTWSKRQLYSALANKCCHVGCTKRS
LARFC.

2. Transformation of the Recombinant Human Relaxin 800800

Five to 10 μg of the relaxin 800800 expression vector that was obtained from the above steps was linearized with SalI (Takata, Cat. D1080a), and 1/10 volume of 3M sodium acetate and 2 volumes of anhydrous ethanol were added. The mixture was placed in −20° C. for 2 hr. The mixture was centrifuged at high speed (13,000 rpm) for 5 min, the supernatant was removed, and the pellet was rinsed with 75% ethanol twice. The pellet was dried by inverting the tube, and then dissolved in 10 μL of ddH₂O. The linearized plasmid and 80 μL of electroporation competent cells (Pichia GS 115, Invitrogen, Cat. K1750-01) were mixed and loaded onto the electroporation cuvette (Bio Rad, Cat. 1652086) for 5 minutes in an ice bath. The electroporation was performed using the electroporation device (Bio Rad Micropulser) with the parameters of 2 kV, 25Ω, and 200 uF. 1 ml of ice-bath D-sorbitol (Bioengineering Co., Ltd.) was then added rapidly to the cuvette and mixed. 100 to 300 μl of the mixture were spread onto a Minimal Dextrose (MD) plate, and the plate was incubated for 3 days at 30° C. until colonies were observed.

3. G418 Selection of Recombinant Human Relaxin 800800 Expression Clone

Colonies on the MD culture plate were eluted with 3 ml of Yeast Extract Peptone Dextrose Medium (YPD) medium and re-suspended, and the concentration of re-suspended cells was measured (1 OD₆₀₀=5×10⁷ cell/ml) by a spectrophotometer (Beckman, DU800). 1×10⁵ cells were spread on a YPD plate containing 4 mg/ml of G418 antibiotic (GIBCO, Cat. 11811-031), and the plate was incubated for 5 days at 30° C. until colonies were observed.

4. Inducible Expression of Recombinant Human Relaxin 800800

A single colony was picked from a YPD plate, placed in 4 mL of Buffered Glycerol-complex (BMGY) medium and cultured overnight at 30° C., shaking at 250 rpm. The culture was measured for its OD₆₀₀ value the next day to ensure that value was between 2 and 6. Cells were collected by centrifuge (Beckman Coulter) at a low speed (1,500 g) for 5 min at room temperature, and resuspended in BMMY medium until the OD600 was 1.0. 1/200 volume of 100% methanol (final concentration of 0.5%) was added, and the culture was incubated at 28° C., shaking at 250 rpm for 72 hr. A supplement of 1/200 of 100% methanol was added every 24 hrs. After induction, the mixture was centrifuged at low-speed (1,500 g) and the supernatant was collected. The protein expression was detected by SDS-PAGE electrophoresis (Invitrogen, Cat. No. 456-1083), and positive clones were selected for the subsequent fermentation step.

The sequences of the resulting recombinant human relaxin 800800 (WT, wild-type) chains were as follows:

chain B (WT)

SEQ ID NO: 134

DSWMEEVIKLCGRELVRAQIAICGMSTWS
chain A (WT)

SEQ ID NO: 135

QLYSALANKCCHVGCTKRSLARFC.

### Example 2

### Cloning and Expression of Recombinant Human Relaxin 800801

1. Construction of Recombinant Human Relaxin 800801 Expression Vector

The DNA sequence of relaxin 800801 was synthesized by site-directed PCR mutagenesis. Two single-stranded DNA fragments synthesized by Invitrogen were used as site-directed mutagenesis primers, and their sequences were as follows:

801-Primer 1

SEQ ID NO: 43

GTATGTCTACTTGGTCTAAGAGATCTTTGAAGAGACAATTGTACTC
801-Primer 2

SEQ ID NO: 44

GAGTACAATTGTCTCTTCAAAGATCTCTTAGACCAAGTAGACATAC

A vector comprising the DNA sequence of relaxin 800800 was used as a template for site-directed PCR mutagenesis using the KOD plus kit (TOYOBO, Cat KOD-201) and a 25 μL reaction volume (2.5 μL of 10×KOD buffer, 2.5 μL of 2 mM dNTPs, 1 μL each of primers 1 and 2 (10 μM), 0.5 μL of KOD plus, 1 μL of 25 mM MgSO4, 16.5 μL of ddH₂O). The thermocycling program was as follows: one cycle of 94° C. for 5 minutes; 30 amplification cycles of 94° C. for 30 seconds, 60° C. for 30 seconds, and 68° C. for 12 minutes; then one cycle of 68° C. for 12 minutes to terminate PCR amplification. The resulting PCR product was digested for 5 hours with 1 μL of endonuclease DpnI (NEB Cat. R0176L), and the digested product was transformed into DH5a competent cells (Tiangen, Cat. CB101-02). Positive clones were picked, and sequenced by Invitrogen. The DNA Sequence of 800801 precursor is as follows:

SEQ ID NO: 45

GATTCTTGGATGGAAGAAGTTATTAAGTTGTGTGGTAGAGAATTGGTTA
GAGCTCAAATTGCTATTTGTGGTATGTCTACTTGGTCTAAGAGATCTTT
GAAGAGACAATTGTACTCTGCTTTGGCTAACAAGTGTTGTCATGTTGG
TTGTACTAAGAGATCTTTGGCTAGATTTTGTTAA

The amino acid sequence coded by the DNA sequence above is as follows:

SEQ ID NO: 2

DSWMEEVIKLCGRELVRAQIAICGMSTWSKRSLKRQLYSALANKCCHVGC
TKRSLARFC

2. Transformation, Screening and Inducible Expression of the Recombinant Human Relaxin 800801

The procedure for the transformation, screening and inducible expression of the recombinant human relaxin 800801 was the same as that described in Example 1.

The sequences of the resulting recombinant human relaxin 800801 (WT, wild-type) chains were as follows:

chain B (WT)

SEQ ID NO: 134

DSWMEEVIKLCGRELVRAQIAICGMSTWS
chain A (WT)

SEQ ID NO: 135

QLYSALANKCCHVGCTKRSLARFC.

### Example 3

### Cloning and Expression of Recombinant Human Relaxin 800802

1. Construction of Recombinant Human Relaxin 800802 Expression Vector

The DNA sequence of relaxin 800802 was synthesized by site-directed PCR mutagenesis. Two single-stranded DNA fragments synthesized by Invitrogen were used as site-directed mutagenesis primers, and their sequences were as follows:

802- Primer 1

SEQ ID NO: 46

GTCTACTTGGTCTAAGAGAAAGCCAACTGGTTACGGTTCTAGAAAGAA
GAGACAATTGTACTCTGC
802- Primer 2

SEQ ID NO: 47

GCAGAGTACAATTGTCTCTTCTTTCTAGAACCGTAACCAGTTGGCTTTC
TCTTAGACCAAGTAGAC

A vector comprising the DNA sequence of relaxin 800801 was used as a template for site-directed PCR mutagenesis using the KOD plus kit (TOYOBO, Cat KOD-201) and a 25 μL reaction volume (2.5 μL of 10×KOD buffer, 2.5 μL of 2 mM dNTPs, 1 μL each of primers 1, 2 (10 μM), 0.5 μL of KOD plus, 1 μL of 25 mM MgSO4, 16.5 μL of ddH₂O). The thermocycling program was as follows: one cycle of 94° C. for 5 minutes; 30 amplification cycles of 94° C. for 30 seconds, 60° C. for 30 seconds, and 68° C. for 12 minutes; then one cycle of 68° C. for 12 minutes to terminate PCR amplification. The resulting PCR product was digested for 5 hours with 1 μL of endonuclease DpnI (NEB Cat. R0176L), and the digested product was transformed into DH5a competent cells (Tiangen, Cat. CB101-02). Positive clones were picked, and sequenced by Invitrogen. The DNA Sequence of 800802 precursor is as follows:

SEQ ID NO: 48

GATTCTTGGATGGAAGAAGTTATTAAGTTGTGTGGTAGAGAATTGGTTAG
AGCTCAAATTGCTATTTGTGGTATGTCTACTTGGTCTAAGAGAAAGCCAA
CTGGTTACGGTTCTAGAAAGAAGAGACAATTGTACTCTGCTTTGGCTAAC
AAGTGTTGTCATGTTGGTTGTACTAAGAGATCTTTGGCTAGATTTTGTTA
A

The amino acid sequence coded by the DNA sequence above is as follows:

SEQ ID NO: 3

DSWMEEVIKLCGRELVRAQIAICGMSTWSKRKPTGYGSRKKRQLYSALAN
KCCHVGCTKRSLARFC.

2. Transformation, Screening and Inducible Expression of the Recombinant Human Relaxin 800802

The procedure for the transformation, screening and inducible expression of the recombinant human relaxin 800802 was the same as that described in Example 1.

The sequences of the resulting recombinant human relaxin 800802 (WT, wild-type) chains were as follows:

SEQ ID NO: 134

chain B (WT) DSWMEEVIKLCGRELVRAQIAICGMSTWS

SEQ ID NO: 135

chain A (WT) QLYSALANKCCHVGCTKRSLARFC.

### Example 4

### Cloning and Expression of Recombinant Human Relaxin 800802-1

1. Construction of Recombinant Human Relaxin 800802-1 Expression Vector

The DNA sequence of relaxin 800802-1 was synthesized by site-directed PCR mutagenesis. Four single-stranded DNA fragments synthesized by Invitrogen were used as site-directed mutagenesis primers, and their sequences were as follows:

802-1- Primer 1

SEQ ID NO: 49

GTTATTAAGTTGTGTGGTAGAGATTTGGTTAGAGCTCAAATTG
802-1- Primer 2

SEQ ID NO: 50

CAATTTGAGCTCTAACCAAATCTCTACCACACAACTTAATAAC
802-1- Primer 3

SEQ ID NO: 51

GTTCTAGAAAGAAGAGAGATTTGTACTCTGCTTTGG
802-1- Primer 4

SEQ ID NO: 52

CCAAAGCAGAGTACAAATCTCTCTTCTTTCTAGAAC

A vector comprising the DNA sequence of relaxin 800802 was used as a template for site-directed PCR mutagenesis, and the mutation process was the same as that described in Example 2. The DNA Sequence of the 800802-1 precursor is as follows:

SEQ ID NO: 53

GATTCTTGGATGGAAGAAGTTATTAAGTTGTGTGGTAGAGATTTGGTTAG
AGCTCAAATTGCTATTTGTGGTATGTCTACTTGGTCTAAGAGAAAGCCAA
CTGGTTACGGTTCTAGAAAGAAGAGAGATTTGTACTCTGCTTTGGCTAAC
AAGTGTTGTCATGTTGGTTGTACTAAGAGATCTTTGGCTAGATTTTGTTA
A

The amino acid sequence coded by the DNA sequence above is as follows:

SEQ ID NO: 4

DSWMEEVIKLCGRDLVRAQIAICGMSTWSKRKPTGYGSRKKRDLYSALA
NKCCHVGCTKRSLARFC.

2. Transformation, Screening and Inducible Expression of the Recombinant Human Relaxin 800802-1

The procedure for the transformation, screening and inducible expression of the recombinant human relaxin 800802-1 was the same as that described in Example 1.

The sequences of the resulting recombinant human relaxin analog 800802-1 chains were as follows:

SEQ ID NO: 137

chain B (D^{B14}) DSWMEEVIKLCGRDLVRAQIAICGMSTWS

SEQ ID NO: 138

chain A (D^{A1}) DLYSALANKCCHVGCTKRSLARFC.

### Example 5

### Cloning and Expression of Recombinant Human Relaxin 800803

1. Construction of Recombinant Human Relaxin 800803 Expression Vector

The DNA sequence of relaxin 800803 was synthesized by site-directed PCR mutagenesis. Two single-stranded DNA fragments synthesized by Invitrogen were used as site-directed mutagenesis primers, and their sequences were as follows:

803- Primer 1

SEQ ID NO: 54

GTCTACTTGGTCTAAGAGAAAGCCAACTGGTTACGGTTCTAGAAAGAG
ACAATTGTACTCTGC
803- Primer 2

SEQ ID NO: 55

GCAGAGTACAATTGTCTCTTTCTAGAACCGTAACCAGTTGGCTTTCTCT
TAGACCAAGTAGAC

A vector comprising the DNA sequence of relaxin 800801 was used as a template for site-directed PCR mutagenesis, and the mutation process was the same as that described in Example 2. The DNA Sequence of the 800803 precursor is as follows:

SEQ ID NO: 56

GATTCTTGGATGGAAGAAGTTATTAAGTTGTGTGGTAGAGAATTGGTTAG
AGCTCAAATTGCTATTTGTGGTATGTCTACTTGGTCTAAGAGAAAGCCAA
CTGGTTACGGTTCTAGAAAGAGACAATTGTACTCTGCTTTGGCTAACAAG
TGTTGTCATGTTGGTTGTACTAAGAGATCTTTGGCTAGATTTTGTTAA

The amino acid sequence coded by the DNA sequence above is as follows:

SEQ ID NO: 5

DSWMEEVIKLCGRELVRAQIAICGMSTWSKRKPTGYGSRKRQLYSALANK
CCHVGCTKRSLARFC.

2. Transformation, Screening and Inducible Expression of the Recombinant Human Relaxin 800803

The procedure for the transformation, screening and inducible expression of the recombinant human relaxin 800803 was the same as that described in Example 1.

The sequences of the resulting recombinant human relaxin 800803 (WT, wild-type) chains were as follows:

SEQ ID NO: 134

chain B (WT) DSWMEEVIKLCGRELVRAQIAICGMSTWS

SEQ ID NO: 135

chain A (WT) QLYSALANKCCHVGCTKRSLARFC.

### Example 6

### Cloning and Expression of Recombinant Human Relaxin 800803-1

1. Construction of Recombinant Human Relaxin 800803-1 Expression Vector

The DNA sequence of relaxin 800803-1 was synthesized by site-directed PCR mutagenesis. Four single-stranded DNA fragments synthesized by Invitrogen were used as site-directed mutagenesis primers, and their sequences were as follows:

803-1- Primer 1

SEQ ID NO: 57

GTTATTAAGTTGTGTGGTAGAGATTTGGTTAGAGCTCAAATTG
803-1- Primer 2

SEQ ID NO: 58

CAATTTGAGCTCTAACCAAATCTCTACCACACAACTTAATAAC
803-1- Primer 3

SEQ ID NO: 59

CGGTTCTAGAAAGAGAGATTTGTACTCTGCTTTGG
803-1- Primer 4

SEQ ID NO: 60

CCAAAGCAGAGTACAAATCTCTCTTTCTAGAACCG

A vector comprising the DNA sequence of relaxin 800803 was used as a template for site-directed PCR mutagenesis, and the mutation process was the same as that described in Example 2. The DNA Sequence of the 800803-1 precursor is as follows:

SEQ ID NO: 61

GATTCTTGGATGGAAGAAGTTATTAAGTTGTGTGGTAGAGATTTGGTTAG
AGCTCAAATTGCTATTTGTGGTATGTCTACTTGGTCTAAGAGAAAGCCAA
CTGGTTACGGTTCTAGAAAGAGAGATTTGTACTCTGCTTTGGCTAACAAG
TGTTGTCATGTTGGTTGTACTAAGAGATCTTTGGCTAGATTTTGTTAA

The amino acid sequence coded by the DNA sequence above is as follows:

SEQ ID NO: 6

DSWMEEVIKLCGRDLVRAQIAICGMSTWSKRKPTGYGSRKRDLYSALANK
CCHVGCTKRSLARFC.

2. Transformation, Screening and Inducible Expression of the Recombinant Human Relaxin 800803-1

The procedure for the transformation, screening and inducible expression of the recombinant human relaxin 800803-1 was the same as that described in Example 1.

The sequences of the resulting recombinant human relaxin analog 800803-1 chains were as follows:

chain B (D^{B14})

SEQ ID NO: 137

DSWMEEVIKLCGRDLVRAQIAICGMSTWS
chain A (D^{A1})

SEQ ID NO: 138

DLYSALANKCCHVGCTKRSLARFC.

### Example 7

### Cloning and Expression of Recombinant Human Relaxin 800805

1. Construction of Recombinant Human Relaxin 800805 Expression Vector

The DNA sequence of relaxin 800805 was synthesized by site-directed PCR mutagenesis. Two single-stranded DNA fragments synthesized by Invitrogen were used as site-directed mutagenesis primers, and their sequences were as follows:

805- Primer 1

SEQ ID NO: 62

GTCTACTTGGTCTAAGAGAGGTGGTGGTCCAAGAAGACAATTGTACTCT
GCTTTG
805- Primer 2

SEQ ID NO: 63

CAAAGCAGAGTACAATTGTCTTCTTGGACCACCACCTCTCTTAGACCAA
GTAGAC

A vector comprising the DNA sequence of relaxin 800801 was used as a template for site-directed PCR mutagenesis, and the mutation process was the same as that described in Example 2. The DNA Sequence of the 800805 precursor is as follows:

SEQ ID NO: 64

GATTCTTGGATGGAAGAAGTTATTAAGTTGTGTGGTAGAGAATTGGTTA
GAGCTCAAATTGCTATTTGTGGTATGTCTACTTGGTCTAAGAGAGGTGGT
GGTCCAAGAAGACAATTGTACTCTGCTTTGGCTAACAAGTGTTGTCATG
TTGGTTGTACTAAGAGATCTTTGGCTAGATTTTGTTAA

The amino acid sequence coded by the DNA sequence above is as follows:

SEQ ID NO: 7

DSWMEEVIKLCGRELVRAQIAICGMSTWSKRGGGPRRQLYSALANKCCH
VGCTKRSLARFC

2. Transformation, Screening and Inducible Expression of the Recombinant Human Relaxin 800805

The procedure for the transformation, screening and inducible expression of the recombinant human relaxin 800805 was the same as that described in Example 1.

The sequences of the resulting recombinant human relaxin 800805 (WT, wild-type) chains were as follows:

chain B (WT)

SEQ ID NO: 134

DSWMEEVIKLCGRELVRAQIAICGMSTWS
chain A (WT)

SEQ ID NO: 135

QLYSALANKCCHVGCTKRSLARFC.

### Example 8

### Cloning and Expression of Recombinant Human Relaxin 8008054

1. Construction of Recombinant Human Relaxin 800805-1 Expression Vector

The DNA sequence of relaxin 800805-1 was synthesized by site-directed PCR mutagenesis. Four single-stranded DNA fragments synthesized by Invitrogen were used as site-directed mutagenesis primers, and their sequences were as follows:

805-1- Primer 1

SEQ ID NO: 65

GTTATTAAGTTGTGTGGTAGAGATTTGGTTAGAGCTCAAATTG
805-1- Primer 2

SEQ ID NO: 66

CAATTTGAGCTCTAACCAAATCTCTACCACACAACTTAATAAC
805-1- Primer 3

SEQ ID NO: 67

GGTGGTCCAAGAAGAGATTTGTACTCTGCTTTGG
805-1- Primer 4

SEQ ID NO: 68

CCAAAGCAGAGTACAAATCTCTTCTTGGACCACC

A vector comprising the DNA sequence of relaxin 800805 was used as a template for site-directed PCR mutagenesis, and the mutation process was the same as that described in Example 2. The DNA Sequence of the 800805-1 precursor is as follows:

SEQ ID NO: 69

GATTCTTGGATGGAAGAAGTTATTAAGTTGTGTGGTAGAGATTTGGTTA
GAGCTCAAATTGCTATTTGTGGTATGTCTACTTGGTCTAAGAGAGGTGGT
GGTCCAAGAAGAGATTTGTACTCTGCTTTGGCTAACAAGTGTTGTCATG
TTGGTTGTACTAAGAGATCTTTGGCTAGATTTTGTTAA

The amino acid sequence coded by the DNA sequence above is as follows:

SEQ ID NO: 8

DSWMEEVIKLCGRDLVRAQIAICGMSTWSKRGGGPRRDLYSALANKCCH
VGCTKRSLARFC.

2. Transformation, Screening and Inducible Expression of the Recombinant Human Relaxin 800805-1

The procedure for the transformation, screening and inducible expression of the recombinant human relaxin 800805-1 was the same as that described in Example 1.

The sequences of the resulting recombinant human relaxin analog 800805-1 chains were as follows:

chain B (D^{B14})

SEQ ID NO: 137

DSWMEEVIKLCGRDLVRAQIAICGMSTWS
chain A (D^{A1})

SEQ ID NO: 138

DLYSALANKCCHVGCTKRSLARFC.

### Example 9

### Cloning and Expression of Recombinant Human Relaxin 800806

1. Construction of Recombinant Human Relaxin 800806 Expression Vector

The DNA sequence of relaxin 800806 was synthesized by site-directed PCR mutagenesis. Two single-stranded DNA fragments synthesized by Invitrogen were used as site-directed mutagenesis primers, and their sequences were as follows:

806- Primer 1

SEQ ID NO: 70

GTCTACTTGGTCTAAGAGAGGTGGTGGTCCAAAGAGACAATTGTACTCT
GCT
806- Primer 2

SEQ ID NO: 71

AGCAGAGTACAATTGTCTCTTTGGACCACCACCTCTCTTAGACCAAGTA
GAC

A vector comprising the DNA sequence of relaxin 800801 was used as a template for site-directed PCR mutagenesis, and the mutation process was the same as that described in Example 2. The DNA Sequence of the 800806 precursor is as follows:

SEQ ID NO: 72

GATTCTTGGATGGAAGAAGTTATTAAGTTGTGTGGTAGAGAATTGGTTA
GAGCTCAAATTGCTATTTGTGGTATGTCTACTTGGTCTAAGAGAGGTGGT
GGTCCAAAGAGACAATTGTACTCTGCTTTGGCTAACAAGTGTTGTCATGT
TGGTTGTACTAAGAGATCTTTGGCTAGATTTTGTTAA

The amino acid sequence coded by the DNA sequence above is as follows:

SEQ ID NO: 9

DSWMEEVIKLCGRELVRAQIAICGMSTWSKRGGGPKRQLYSALANKCCH
VGCTKRSLARFC.

2. Transformation, Screening and Inducible Expression of the Recombinant Human Relaxin 800806

The procedure for the transformation, screening and inducible expression of the recombinant human relaxin 800806 was the same as that described in Example 1.

The sequences of the resulting recombinant human relaxin 800806 chains (WT, wild-type) were as follows:

chain B (WT)

SEQ ID NO: 134

DSWMEEVIKLCGRELVRAQIAICGMSTWS
chain A (WT)

SEQ ID NO: 135

QLYSALANKCCHVGCTKRSLARFC.

### Example 10

### Cloning and Expression of Recombinant Human Relaxin 800806-1

1. Construction of Recombinant Human Relaxin 800806-1 Expression Vector

The DNA sequence of relaxin 800806-1 was synthesized by site-directed PCR mutagenesis. Four single-stranded DNA fragments synthesized by Invitrogen were used as site-directed mutagenesis primers, and their sequences were as follows:

806-1- Primer 1

SEQ ID NO: 73

GTTATTAAGTTGTGTGGTAGAGATTTGGTTAGAGCTCAAATTG
806-1- Primer 2

SEQ ID NO: 74

CAATTTGAGCTCTAACCAAATCTCTACCACACAACTTAATAAC
806-1- Primer 3

SEQ ID NO: 75

GGTGGTGGTCCAAAGAGAGATTTGTACTCTGCTTTGG
806-1- Primer 4

SEQ ID NO: 76

CCAAAGCAGAGTACAAATCTCTCTTTGGACCACCACC

A vector comprising the DNA sequence of relaxin 800806 was used as a template for site-directed PCR mutagenesis, and the mutation process was the same as that described in Example 2. The DNA Sequence of the 800806-1 precursor is as follows:

SEQ ID NO: 77

GATTCTTGGATGGAAGAAGTTATTAAGTTGTGTGGTAGAGATTTGGTTA
GAGCTCAAATTGCTATTTGTGGTATGTCTACTTGGTCTAAGAGAGGTGGT
GGTCCAAAGAGAGATTTGTACTCTGCTTTGGCTAACAAGTGTTGTCATGT
TGGTTGTACTAAGAGATCTTTGGCTAGATTTTGTTAA

The amino acid sequence coded by the DNA sequence above is as follows:

SEQ ID NO: 10

DSWMEEVIKLCGRDLVRAQIAICGMSTWSKRGGGPKRDLYSALANKCCH
VGCTKRSLARFC

2. Transformation, Screening and Inducible Expression of the Recombinant Human Relaxin 800806-1

The procedure for the transformation, screening and inducible expression of the recombinant human relaxin 800806-1 was the same as that described in Example 1.

The sequences of the resulting recombinant human relaxin analog 800806-1 chains were as follows:

chain B (D^{B14})

SEQ ID NO: 137

DSWMEEVIKLCGRDLVRAQIAICGMSTWS
chain A (D^{A1})

SEQ ID NO: 138

DLYSALANKCCHVGCTKRSLARFC.

### Example 11

### Cloning and Expression of Recombinant Human Relaxin 800808

1. Construction of Recombinant Human Relaxin 800808 Expression Vector

The DNA sequence of relaxin 800808 was synthesized by site-directed PCR mutagenesis. Two single-stranded DNA fragments synthesized by Invitrogen were used as site-directed mutagenesis primers, and their sequences were as follows:

808 - Primer 1

SEQ ID NO: 78

GGGTATCTCTCGAGAAAAGAGAAGAAGGTGAACCAAAGGATTCTTGGA
TGGAAGAAG
808 - Primer 2

SEQ ID NO: 79

CTTCTTCCATCCAAGAATCCTTTGGTTCACCTTCTTCTCTTTTCTCGAGA
GATACCC

A vector comprising the DNA sequence of relaxin 800800 carrier was used as a template for site-directed PCR mutagenesis, and the mutation process was the same as that described in Example 2. The DNA Sequence of the 800808 precursor is as follows:

SEQ ID NO: 80

GAAGAAGGTGAACCAAAGGATTCTTGGATGGAAGAAGTTATTAAGTTG
TGTGGTAGAGAATTGGTTAGAGCTCAAATTGCTATTTGTGGTATGTCTAC
TTGGTCTAAGAGACAATTGTACTCTGCTTTGGCTAACAAGTGTTGTCA
TGTTGGTTGTACTAAGAGATCTTTGGCTAGATTTTGTTAA

The amino acid sequence coded by the DNA sequence above is as follows:

SEQ ID NO: 11

EEGEPKDSWMEEVIKLCGRELVRAQIAICGMSTWSKRQLYSALANKCCHV
GCTKRSLARFC

2. Transformation, Screening and Inducible Expression of the Recombinant Human Relaxin 800808

the procedure for the transformation, screening and inducible expression of the recombinant human relaxin 800808 was the same as that described in Example 1.

The sequences of resulting the recombinant human relaxin 800808 chains (WT, wild-type) were as follows:

chain B (WT)

SEQ ID NO: 134

DSWMEEVIKLCGRELVRAQIAICGMSTWS
chain A (WT)

SEQ ID NO: 135

QLYSALANKCCHVGCTKRSLARFC.

### Example 12

### Cloning and Expression of Recombinant Human Relaxin 800808-1

1. Construction of Recombinant Human Relaxin 800808-1 Expression Vector

The DNA sequence of relaxin 800808-1 was synthesized by site-directed PCR mutagenesis. Four single-stranded DNA fragments synthesized by Invitrogen were used as site-directed mutagenesis primers, and their sequences were as follows:

808-1 - Primer 1

SEQ ID NO: 81

GTTATTAAGTTGTGTGGTAGAGATTTGGTTAGAGCTCAAATTG
808-1 - Primer 2

SEQ ID NO: 82

CAATTTGAGCTCTAACCAAATCTCTACCACACAACTTAATAAC
808-1 - Primer 3

SEQ ID NO: 83

CTACTTGGTCTAAGAGAGATTTGTACTCTGCTTTGG
808-1 - Primer 4

SEQ ID NO: 84

CCAAAGCAGAGTACAAATCTCTCTTAGACCAAGTAG

A vector comprising the DNA sequence of relaxin 800808 was used as a template for site-directed PCR mutagenesis, and the mutation process was the same as that described in Example 2. The DNA Sequence of the 800808-1 precursor is as follows:

SEQ ID NO: 85

GAAGAAGGTGAACCAAAGGATTCTTGGATGGAAGAAGTTATTAAGTTG
TGTGGTAGAGATTTGGTTAGAGCTCAAATTGCTATTTGTGGTATGTCTA
CTTGGTCTAAGAGAGATTTGTACTCTGCTTTGGCTAACAAGTGTTGTCAT
GTTGGTTGTACTAAGAGATCTTTGGCTAGATTTTGTTAA
The amino acid sequence coded by the DNA
sequence above is as follows:

SEQ ID NO: 12

EEGEPKDSWMEEVIKLCGRDLVRAQIAICGMSTWSKRDLYSALANKCCHV
GCTKRSLARFC

2. Transformation, Screening and Inducible Expression of the Recombinant Human Relaxin 800808-1

The procedure for the transformation, screening and inducible expression of the recombinant human relaxin 800808-1 was the same as that described in Example 1.

The sequences of the resulting recombinant human relaxin analog 800808-1 chains were as follows:

chain B (D^{B14})

SEQ ID NO: 137

DSWMEEVIKLCGRDLVRAQIAICGMSTWS
chain A (D^{A1})

SEQ ID NO: 138

DLYSALANKCCHVGCTKRSLARFC.

### Example 13

### Cloning and Expression of Recombinant Human Relaxin 800809

1. Construction of Recombinant Human Relaxin 800809 Expression Vector

The DNA sequence of relaxin 800809 was synthesized by site-directed PCR mutagenesis. Two single-stranded DNA fragments synthesized by Invitrogen were used as site-directed mutagenesis primers, and their sequences were as follows:

809 - Primer 1

SEQ ID NO: 86

GGGTATCTCTCGAGAAAAGAGAAGAAGGTGAACCAAAGAGAGATTCTT
GGATGGAAGAAG
809 - Primer 2

SEQ ID NO: 87

CTTCTTCCATCCAAGAATCTCTCTTTGGTTCACCTTCTTCTCTTTTCTC
GAGAGATACCC

A vector comprising the DNA sequence of relaxin 800800 was used as a template for site-directed PCR mutagenesis, and the mutation process was the same as that described in Example 2. The DNA Sequence of the 800809 precursor is as follows:

SEQ ID NO: 88

GAAGAAGGTGAACCAAAGAGAGATTCTTGGATGGAAGAAGTTATTAAG
TTGTGTGGTAGAGAATTGGTTAGAGCTCAAATTGCTATTTGTGGTATGT
CTACTTGGTCTAAGAGACAATTGTACTCTGCTTTGGCTAACAAGTGTTG
TCATGTTGGTTGTACTAAGAGATCTTTGGCTAGATTTTGTTAA

The amino acid sequence coded by the DNA sequence above is as follows:

SEQ ID NO: 13

EEGEPKRDSWMEEVIKLCGRELVRAQIAICGMSTWSKRQLYSALANKCCH
VGCTKRSLARFC.

2. Transformation, Screening and Inducible Expression of the Recombinant Human Relaxin 800809

The procedure for the transformation, screening and inducible expression of the recombinant human relaxin 800809 was the same as that described in Example 1.

The sequences of the resulting recombinant human relaxin 800809 chains (WT, wild-type) were as follows:

chain B (WT)

SEQ ID NO: 134

DSWMEEVIKLCGRELVRAQIAICGMSTWS
chain A (WT)

SEQ ID NO: 135

QLYSALANKCCHVGCTKRSLARFC.

### Example 14

### Cloning and Expression of Recombinant Human Relaxin 800809-1

1. Construction of Recombinant Human Relaxin 800809-1 Expression Vector

The DNA sequence of relaxin 800809-1 was synthesized by site-directed PCR mutagenesis. Four single-stranded DNA fragments synthesized by Invitrogen were used as site-directed mutagenesis primers, and their sequences were the same as those for 800808-1:

809-1 - Primer 1

SEQ ID NO: 89

GTTATTAAGTTGTGTGGTAGAGATTTGGTTAGAGCTCAAATTG
809-1 - Primer 2

SEQ ID NO: 90

CAATTTGAGCTCTAACCAAATCTCTACCACACAACTTAATAAC
809-1 - Primer 3

SEQ ID NO: 91

CTACTTGGTCTAAGAGAGATTTGTACTCTGCTTTGG
809-1 - Primer 4

SEQ ID NO: 92

CCAAAGCAGAGTACAAATCTCTCTTAGACCAAGTAG

A vector comprising the DNA sequence of relaxin 800809 carrier was used as a template site-directed PCR mutagenesis, and the mutation process was the same as that described in Example 2. The DNA Sequence of the 800809-1 precursor is as follows:

SEQ ID NO: 93

GAAGAAGGTGAACCAAAGAGAGATTCTTGGATGGAAGAAGTTATTAAG
TTGTGTGGTAGAGATTTGGTTAGAGCTCAAATTGCTATTTGTGGTATGT
CTACTTGGTCTAAGAGAGATTTGTACTCTGCTTTGGCTAACAAGTGTTGT
CATGTTGGTTGTACTAAGAGATCTTTGGCTAGATTTTGTTAA

The amino acid sequence coded by the DNA sequence above is as follows:

SEQ ID NO: 14

EEGEPKRDSWMEEVIKLCGRDLVRAQIAICGMSTWSKRDLYSALANKCCH
VGCTKRSLARFC.

2. Transformation, Screening and Inducible Expression of the Recombinant Human Relaxin 800809-1

The procedure for the transformation, screening and inducible expression of the recombinant human relaxin 800809-1 was the same as that described in Example 1.

The sequences of the resulting recombinant human relaxin analog 800809-1 chains were as follows:

SEQ ID NO: 137

chain B (D^{B14}) DSWMEEVIKLCGRDLVRAQIAICGMSTWS

SEQ ID NO: 138

chain A (D^{A1}) DLYSALANKCCHVGCTKRSLARFC.

### Example 15

### Cloning and Expression of Recombinant Human Relaxin 800810

1. Construction of Recombinant Human Relaxin 800810 Expression Vector

The DNA sequence of relaxin 800810 was synthesized by site-directed PCR mutagenesis. Two single-stranded DNA fragments synthesized by Invitrogen were used as site-directed mutagenesis primers, and their sequences were as follows:

810- Primer 1

SEQ ID NO: 94

GTTATTAAGTTGTGTGGTAGAGATTTGGTTAGAGCTCAAATTG
810- Primer 2

SEQ ID NO: 95

CAATTTGAGCTCTAACCAAATCTCTACCACACAACTTAATAAC

A vector comprising the DNA sequence of relaxin 800800 carrier was used as a template for site-directed PCR mutagenesis, and the mutation process was the same as that described in Example 2. The DNA Sequence of the 800810 precursor is as follows:

SEQ ID NO: 96

GATTCTTGGATGGAAGAAGTTATTAAGTTGTGTGGTAGAGATTTGGTTAG
AGCTCAAATTGCTATTTGTGGTATGTCTACTTGGTCTAAGAGACAATTGT
ACTCTGCTTTGGCTAACAAGTGTTGTCATGTTGGTTGTACTAAGAGATCT
TTGGCTAGATTTTGTTAA

The amino acid sequence coded by the DNA sequence above is as follows:

SEQ ID NO: 15

DSWMEEVIKLCGRDLVRAQIAICGMSTWSKRQLYSALANKCCHVGCTKRS
LARFC.

2. Transformation, Screening and Inducible Expression of the Recombinant Human Relaxin 800810

The procedure for the transformation, screening and inducible expression of the recombinant human relaxin 800810 was the same as that described in Example 1.

The sequences of the resulting recombinant human relaxin 800810 chains were as follows:

SEQ ID NO: 137

chain B (D^{B14}) DSWMEEVIKLCGRDLVRAQIAICGMSTWS

SEQ ID NO: 135

chain A (WT) QLYSALANKCCHVGCTKRSLARFC.

### Example 16

### Cloning and Expression of Recombinant Human Relaxin 800810-1

1. Construction of Recombinant Human Relaxin 800810-1 Expression Vector

The DNA sequence of relaxin 800810-1 was synthesized by site-directed PCR mutagenesis. Two single-stranded DNA fragments synthesized by Invitrogen were used as site-directed mutagenesis primers, and their sequences were as follows:

810-1- Primer 1

SEQ ID NO: 97

CTACTTGGTCTAAGAGAGATTTGTACTCTGCTTTGG
810-1- Primer 2

SEQ ID NO: 98

CCAAAGCAGAGTACAAATCTCTCTTAGACCAAGTAG

A vector comprising the DNA sequence of relaxin 800810 was used as a template for site-directed PCR mutagenesis, and the mutation process was the same as that described in Example 2. The DNA Sequence of the 800810-1 precursor is as follows:

SEQ ID NO: 99

GATTCTTGGATGGAAGAAGTTATTAAGTTGTGTGGTAGAGATTTGGTTAG
AGCTCAAATTGCTATTTGTGGTATGTCTACTT6GGTCTAAGAGAGATTTG
TACTCTGCTTTGGCTAACAAGTGTTGTCATGTTGGTTGTACTAAGAGATC
TTTGGCTAGATTTTGTTAA

The amino acid sequence coded by the DNA sequence above is as follows:

SEQ ID NO: 16

DSWMEEVIKLCGRDLVRAQIAICGMSTWSKRDLYSALANKCCHVGCTKRS
LARFC.

2. Transformation, Screening and Inducible Expression of the Recombinant Human Relaxin 800810-1

The procedure for the transformation, screening and inducible expression of the recombinant human relaxin 800810-1 was the same as that described in Example 1.

The sequences of the resulting recombinant human relaxin analog 800810-1 chains were as follows:

SEQ ID NO: 137

chain B (D^{B14}) DSWMEEVIKLCGRDLVRAQIAICGMSTWS

SEQ ID NO: 138

chain A (D^{A1}) DLYSALANKCCHVGCTKRSLARFC.

### Example 17

### Cloning and Expression of Recombinant Human Relaxin 800811

1. Construction of Recombinant Human Relaxin 800811 Expression Vector

The DNA sequence of relaxin 800811 was synthesized by site-directed PCR mutagenesis. Two single-stranded DNA fragments synthesized by Invitrogen were used as site-directed mutagenesis primers, and their sequences were as follows:

811- Primer 1

SEQ ID NO: 100

GTTATTAAGTTGTGTGGTAGAGATTTGGTTAGAGCTCAAATTG
811- Primer 2

SEQ ID NO: 101

CAATTTGAGCTCTAACCAAATCTCTACCACACAACTTAATAAC

A vector comprising the DNA sequence of relaxin 800804 was used as a template for site-directed PCR mutagenesis, and the mutation process was the same as that described in Example 2. The DNA Sequence of the 800811 precursor is as follows:

SEQ ID NO: 102

GATTCTTGGATGGAAGAAGTTATTAAGTTGTGTGGTAGAGATTTGGTTAG
AGCTCAAATTGCTATTTGTGGTATGTCTACTTGGTCTAAGAGAAAGCCAA
CTGGTTACGGTTCTAAGAGACAATTGTACTCTGCTTTGGCTAACAAGTGT
TGTCATGTTGGTTGTACTAAGAGATCTTTGGCTAGATTTTGTTAA

The amino acid sequence coded by the DNA sequence above is as follows:

SEQ ID NO: 17

DSWMEEVIKLCGRDLVRAQIAICGMSTWSKRKPTGYGSKRQLYSALANKC
CHVGCTKRSLARFC.

2. Transformation, Screening and Inducible Expression of the Recombinant Human Relaxin 800811

The procedure for the transformation, screening and inducible expression of the recombinant human relaxin 800811 was the same as that described in Example 1.

The sequences of the resulting recombinant human relaxin analog 800811 chains were as follows:

SEQ ID NO: 137

chain B (D^{B14}) DSWMEEVIKLCGRDLVRAQIAICGMSTWS

SEQ ID NO: 135

chain A (WT) QLYSALANKCCHVGCTKRSLARFC.

### Example 18

### Cloning and Expression of Recombinant Human Relaxin 800813

1. Construction of Recombinant Human Relaxin 800813 Expression Vector

The DNA sequence of relaxin 800813 was synthesized by site-directed PCR mutagenesis. Two single-stranded DNA fragments synthesized by Invitrogen were used as site-directed mutagenesis primers, and their sequences were as follows:

813- Primer 1

SEQ ID NO: 103

CTGGTTACGGTTCTAAGAGAGAATTGTACTCTGCTTTGGC
813- Primer 2

SEQ ID NO: 104

GCCAAAGCAGAGTACAATTCTCTCTTAGAACCGTAACCAG

A vector comprising the DNA sequence of relaxin 800811 was used as a template for site-directed PCR mutagenesis, and the mutation process was the same as that described in Example 2. The DNA Sequence of the 800813 precursor is as follows:

SEQ ID NO: 105

GATTCTTGGATGGAAGAAGTTATTAAGTTGTGTGGTAGAGATTTGGTTAG
AGCTCAAATTGCTATTTGTGGTATGTCTACTTGGTCTAAGAGAAAGCCAA
CTGGTTACGGTTCTAAGAGAGAATTGTACTCTGCTTTGGCTAACAAGTGT
TGTCATGTTGGTTGTACTAAGAGATCTTTGGCTAGATTTTGTTAA

The amino acid sequence coded by the DNA sequence above is as follows:

SEQ ID NO: 18

DSWMEEVIKLCGRDLVRAQIAICGMSTWSKRKPTGYGSKRELYSALANKC
CHVGCTKRSLARFC.

2. Transformation, Screening and Inducible Expression of the Recombinant Human Relaxin 800813

The procedure for the transformation, screening and inducible expression of the recombinant human relaxin 800813 was the same as that described in Example 1.

The sequences of the resulting recombinant human relaxin analog 800813 chains were as follows:

chain B (D^{B14})

SEQ ID NO: 137

DSWMEEVIKLCGRDLVRAQIAICGMSTWS
chain A (E^{A1})

SEQ ID NO: 136

ELYSALANKCCHVGCTKRSLARFC.

### Example 19

### Cloning and Expression of Recombinant Human Relaxin 800814

1. Construction of Recombinant Human Relaxin 800814 Expression Vector

The DNA sequence of relaxin 800814 was synthesized by site-directed PCR mutagenesis. Two single-stranded DNA fragments synthesized by Invitrogen were used as site-directed mutagenesis primers, and their sequences were as follows:

814 - Primer 1

SEQ ID NO: 106

CTGGTTACGGTTCTAAGAGAGATTTGTACTCTGCTTTGGC
814 - Primer 2

SEQ ID NO: 107

GCCAAAGCAGAGTACAAATCTCTCTTAGAACCGTAACCAG

A vector comprising the DNA sequence of relaxin 800811 was used as a template for site-directed PCR mutagenesis, and the mutation process was the same as that described in Example 2. The DNA Sequence of the 800814 precursor is as follows:

SEQ ID NO: 108

GATTCTTGGATGGAAGAAGTTATTAAGTTGTGTGGTAGAGATTTGGTTAG
AGCTCAAATTGCTATTTGTGGTATGTCTACTTGGTCTAAGAGAAAGCCAA
CTGGTTACGGTTCTAAGAGAGATTTGTACTCTGCTTTGGCTAACAAGTGT
TGTCATGTTGGTTGTACTAAGAGATCTTTGGCTAGATTTTGTTAA

The amino acid sequence coded by the DNA sequence above is as follows:

SEQ ID NO: 19

DSWMEEVIKLCGRDLVRAQIAICGMSTWSKRKPTGYGSKRDLYSALANKC
CHVGCTKRSLARFC.

2. Transformation, Screening and Inducible Expression of the Recombinant Human Relaxin 800814

The procedure for the transformation, screening and inducible expression of the recombinant human relaxin 800814 was the same as that described in Example 1.

The sequences of the resulting recombinant human relaxin analog 800814 chains were as follows:

chain B (D^{B14})

SEQ ID NO: 137

DSWMEEVIKLCGRDLVRAQIAICGMSTWS
chain A (D^{A1})

SEQ ID NO: 138

DLYSALANKCCHVGCTKRSLARFC.

### Example 20

### Cloning and Expression of Recombinant Human Relaxin 800816

1. Construction of Recombinant Human Relaxin 800816 Expression Vector

The DNA sequence of relaxin 800816 was synthesized by site-directed PCR mutagenesis. Two single-stranded DNA fragments synthesized by Invitrogen were used as site-directed mutagenesis primers, and their sequences were as follows:

816 - Primer 1

SEQ ID NO: 109

ATTAAGTTGTGTGGTAGAAACTTGGTTAGAGCTCAAATTGC
816 - Primer 2

SEQ ID NO: 110

GCAATTTGAGCTCTAACCAAGTTTCTACCACACAACTTAAT

A vector comprising the DNA sequence of relaxin 800804 was used as a template for site-directed PCR mutagenesis, and the mutation process was the same as that described in Example 2. The DNA Sequence of the 800816 precursor is as follows:

SEQ ID NO: 111

GATTCTTGGATGGAAGAAGTTATTAAGTTGTGTGGTAGAAACTTGGTTAG
AGCTCAAATTGCTATTTGTGGTATGTCTACTTGGTCTAAGAGAAAGCCAA
CTGGTTACGGTTCTAAGAGACAATTGTACTCTGCTTTGGCTAACAAGTGT
TGTCATGTTGGTTGTACTAAGAGATCTTTGGCTAGATTTTGTTAA

The amino acid sequence coded by the DNA sequence above is as follows:

SEQ ID NO: 20

DSWMEEVIKLCGRNLVRAQIAICGMSTWSKRKPTGYGSKRQLYSALANKC
CHVGCTKRSLARFC.

2. Transformation, Screening and Inducible Expression of the Recombinant Human Relaxin 800816

The procedure for the transformation, screening and inducible expression of the recombinant human relaxin 800816 was the same as that described in Example 1.

The sequences of the resulting recombinant human relaxin analog 800816 chains were as follows:

chain B (N^{B14})

SEQ ID NO: 139

DSWMEEVIKLCGRNLVRAQIAICGMSTWS
chain A (WT)

SEQ ID NO: 135

QLYSALANKCCHVGCTKRSLARFC.

### Example 21

### Cloning and Expression of Recombinant Human Relaxin 800847Y

1. Construction of Recombinant Human Relaxin 800847Y Expression Vector

The DNA sequence of relaxin 800847Y was synthesized by site-directed PCR mutagenesis. Two single-stranded DNA fragments synthesized by Invitrogen were used as site-directed mutagenesis primers, and their sequences were as follows:

847Y - Primer 1

SEQ ID NO: 112

AAGTTGTGTGGTAGAGAATTGGTTAGAGCTCAAA
847Y - Primer 2

SEQ ID NO: 113

TTTGAGCTCTAACCAATTCTCTACCACACAACTT

A vector comprising the DNA sequence of relaxin 800814 was used as a template for site-directed PCR mutagenesis, and the mutation process was the same as that described in Example 2. The DNA Sequence of the 800847Y precursor is as follows:

SEQ ID NO: 114

GATTCTTGGATGGAAGAAGTTATTAAGTTGTGTGGTAGAGAATTGGTTAG
AGCTCAAATTGCTATTTGTGGTATGTCTACTTGGTCTAAGAGAAAGCCAA
CTGGTTACGGTTCTAAGAGAGATTTGTACTCTGCTTTGGCTAACAAGTGT
TGTCATGTTGGTTGTACTAAGAGATCTTTGGCTAGATTTTGTTAA

The amino acid sequence coded by the DNA sequence above is as follows:

SEQ ID NO: 21

DSWMEEVIKLCGRELVRAQIAICGMSTWSKRKPTGYGSKRDLYSALANKC
CHVGCTKRSLARFC

2. Transformation, Screening and Inducible Expression of the Recombinant Human Relaxin 800847Y

The procedure for the transformation, screening and inducible expression of the recombinant human relaxin 800847Y was the same as that described in Example 1.

The sequences of the resulting recombinant human relaxin analog 800847Y chains were as follows:

chain B (WT)

SEQ ID NO: 134

DSWMEEVIKLCGRELVRAQIAICGMSTWS
chain A (D^{A1})

SEQ ID NO: 138

DLYSALANKCCHVGCTKRSLARFC.

### Example 22

### Cloning and Expression of Recombinant Human Relaxin 800851Y

1. Construction of Recombinant Human Relaxin 800851Y Expression Vector

The DNA sequence of relaxin 800851Y was synthesized by site-directed PCR mutagenesis. Two single-stranded DNA fragments synthesized by Invitrogen were used as site-directed mutagenesis primers, and their sequences were as follows:

851Y- Primer 1

SEQ ID NO: 115

GGGGTATCTCTCGAGAAAAGATGGATGGAAGAAGTTATTAAG
851Y- Primer 2

SEQ ID NO: 116

CTTAATAACTTCTTCCATCCATCTTTTCTCGAGAGATACCCC

A vector comprising the DNA sequence of relaxin 800814 was used as a template of PCR site-directed mutagenesis, and the mutation process was the same as that described in Example 2. The DNA Sequence of the 800851Y precursor is as follows:

SEQ ID NO: 117

TGGATGGAAGAAGTTATTAAGTTGTGTGGTAGAGATTTGGTTAGAGCTC
AAATTGCTATTTGTGGTATGTCTACTTGGTCTAAGAGAAAGCCAACTGGT
TACGGTTCTAAGAGAGATTTGTACTCTGCTTTGGCTAACAAGTGTTGTC
ATGTTGGTTGTACTAAGAGATCTTTGGCTAGATTTTGTTAA

The amino acid sequence coded by the DNA sequence above is as follows:

SEQ ID NO: 22

WMEEVIKLCGRDLVRAQIAICGMSTWSKRKPTGYGSKRDLYSALANKCC
HVGCTKRSLARFC.

2. Transformation, Screening and Inducible Expression of the Recombinant Human Relaxin 800851Y

The procedure for the transformation, screening and inducible expression of the recombinant human relaxin 80085IY was the same as that described in Example 1.

The sequences of the resulting recombinant human relaxin analog 800851Y chains were as follows:

chain B (D^{B14})

SEQ ID NO: 137

DSWMEEVIKLCGRDLVRAQIAICGMSTWS
chain A (D^{A1})

SEQ ID NO: 138

DLYSALANKCCHVGCTKRSLARFC.

### Example 23

### Cloning and Expression of Recombinant Human Relaxin 800828

1. Construction of Recombinant Human Relaxin 800828 Expression Vector

The full length DNA sequence of codon-optimized human relaxin was synthesized by an Overlapping PCR method by introducing an NdeI cleavage site (CATATG) into the 5′ terminus and a BamHI cleavage site (GGATCC) into the 3′ terminus. Six single-stranded DNA fragments synthesized by Invitrogen were used as synthetic primers, and their sequences are as follows:

828- Primer 1

SEQ ID NO: 118

CATATGAAGAAAAACATCGCGTTCCTGCTGAAACGTGACTCTTGGAT
GGA
828- Primer 2

SEQ ID NO: 119

GCACGAACCAGTTCACGACCGCACAGTTTGATAACTTCTTCCATCC
AAGAGTCACGTTT
828- Primer 3

SEQ ID NO: 120

CGTGAACTGGTTCGTGCGCAAATTGCGATCTGCGGTATGTCTACCT
GGTCTAAACGTAA
828- Primer 4

SEQ ID NO: 121

CAGCTGACGTTTTTTACGAGAACCGTAACCGGTCGGTTTACGTTTA
GACCAGGTAGACA
828- Primer 5

SEQ ID NO: 122

CTCGTAAAAAACGTCAGCTGTACTCTGCGCTGGCGAACAAATGCTG
CCACGTTGGTTGC
828- Primer 6

SEQ ID NO: 123

GGATCCTTAGCAGAAACGCGCCAGAGAACGTTTGGTGCAACCAAC
GTGGCAG

Relaxin was synthesized using a KOD plus kit (TOYOBO, Cat. KOD-201), and the reaction was performed by two-step PCR.

The conditions of PCR step 1 were as follows—

25 μL reaction volume: 2.5 μL of 10×KOD buffer, 2.5 μL of 2 mM dNTPs, 1 μL each of primers 1, 2, 3, 4, 5, 6 (10 μM), 0.5 μL of KOD plus, 1 μL of 25 mM MgSO₄, 12.5 μL of ddH₂O.

Thermocycling program: one cycle of 94° C. for 5 minutes; 30 amplification cycles of 94° C. for 30 seconds, 60° C. for 30 seconds, and 68° C. for 30 seconds; then one cycle of 68° C. for 30 minutes to terminate PCR amplification.

The conditions of PCR step 2 were as follows—

25 μL reaction volume: 2.5 μL of 10×KOD buffer, 2.5 μL of 2 mM dNTPs, 1 μL each of primers 1 and 6 (10 μM), 1 μL of PCR step 1 product, 0.5 μL of KOD plus, 1 μL of 25 mM MgSO₄, 15.5 μL of ddH₂O.

Thermocycling program: one cycle of 94° C. for 5 minutes; 30 amplification cycles of 94° C. for 30 seconds, and 68° C. for 60 seconds; then one cycle of 68° C. for 10 minutes to terminate PCR amplification.

PCR-generated DNA sequences and a pET9a vector (Novagen, Cat. 69431-3) were digested using NdeI and BamHI, respectively (Takara, Cat. D1161A/D1010A). The resulting fragments were recovered by 1.2% agarose gel electrophoresis, ligated using T4 DNA ligase (New England Biolabs, Cat. M0202V), and transformed into DH5a competent cells (Tiangen, Cat. CB101-02). Positive clones were picked, and sequenced by Invitrogen. The DNA sequence of the 800828 precursor is as follows:

SEQ ID NO: 124

CATATGAAGAAAAACATCGCGTTCCTGCTGAAACGTGACTCTTGGATG
GAAGAAGTTATCAAACTGTGCGGTCGTGAACTGGTTCGTGCGCAAATTG
CGATCTGCGGTATGTCTACCTGGTCTAAACGTAAACCGACCGGTTACG
GTTCTCGTAAAAAACGTCAGCTGTACTCTGCGCTGGCGAACAAATGCT
GCCACGTTGGTTGCACCAAACGTTCTCTGGCGCGTTTCTGCTAAGGATCC

The underlined regions indicate restriction endonuclease cleavage sites.

The amino acid sequence coded by the DNA sequence above is as follows:

SEQ ID NO: 23

MKKNIAFLLKRDSWMEEVIKLCGRELVRAQIAICGMSTWSKRKPTGYGSR
KKRQLYSALANKCCHVGCTKRSLARFC.

2. Inducible Expression of Small Amount of Recombinant Human Relaxin 800828

A recombinant PET9a-relaxin 800828 plasmid containing the correct sequence was transformed into BL21 (DE3) competent cells (Tiangen, Cat. CB105-02), and a monoclonal strain was picked for IPTG induction. The specific induction method was as follows: a monoclonal strain was picked from a fresh plate, inoculated into 10 ml LB medium which contained ampicillin, cultured with shaking at 37° C. until the OD600 value reached 0.6. A portion of the sample was collected as a non-induced control and cryopreserved. IPTG was added to the remaining sample from a 1M stock to a final concentration of 1 mM, and the sample was incubated for an additional 4 hours. The sample was harvested by centrifugation and analyzed by SDS-PAGE electrophoresis analysis after the induction.

The sequences of the resulting recombinant human relaxin 800828 chains (WT, wild-type) were as follows:

chain B (WT)

SEQ ID NO: 134

DSWMEEVIKLCGRELVRAQIAICGMSTWS
chain A (WT)

SEQ ID NO: 135

QLYSALANKCCHVGCTKRSLARFC.

### Example 24

### Cloning and Expression of Recombinant Human Relaxin 800843

1. Construction of Recombinant Human Relaxin 800843 Expression Vector

The DNA sequence of relaxin 800843 was synthesized by site-directed PCR mutagenesis. Two single-stranded DNA fragments synthesized by Invitrogen were used as site-directed mutagenesis primers, and their sequences were as follows:

843- Primer 1

SEQ ID NO: 125

GTTCTCGTAAAAAACGTTGGCTGTACTCTGCGCTG
843- Primer 2

SEQ ID NO: 126

CAGCGCAGAGTACAGCCAACGTTTTTTACGAGAAC

A vector comprising the DNA sequence of relaxin 800828 was used as a template for site-directed PCR mutagenesis. The DNA Sequence of the 800843 precursor is as follows:

SEQ ID NO: 127

ATGAAGAAAAACATCGCGTTCCTGCTGAAACGTGACTCTTGGATGGAA
GAAGTTATCAAACTGTGCGGTCGTGAACTGGTTCGTGCGCAAATTGCGAT
CTGCGGTATGTCTACCTGGTCTAAACGTAAACCGACCGGTTACGGTTCTC
GTAAAAAACGTTGGCTGTACTCTGCGCTGGCGAACAAATGCTGCCACGT
TGGTTGCACCAAACGTTCTCTGGCGCGTTTCTGCTAA

The amino acid sequence coded by the DNA sequence above is as follows:

SEQ ID NO: 24

MKKNIAFLLKRDSWMEEVIKLCGRELVRAQIAICGMSTWSKRKPTGYGSR
KKRWLYSALANKCCHVGCTKRSLARFC.

2. Inducible Expression of Small Amount of Recombinant Human Relaxin 800843

The procedure for the inducible expression of a small amount of recombinant human relaxin 800843 was the same as that described in Example 23.

The sequences of the resulting recombinant human relaxin analog 800843 chains were as follows:

chain B (WT)

SEQ ID NO: 134

DSWMEEVIKLCGRELVRAQIAICGMSTWS
chain A (W^{A1})

SEQ ID NO: 141

WLYSALANKCCHVGCTKRSLARFC.

### Example 25

### Cloning and Expression of Recombinant Human Relaxin 800847

1. Construction of Recombinant Human Relaxin 800847 Expression Vector

The DNA sequence of relaxin 800847 was synthesized by site-directed PCR mutagenesis. Two single-stranded DNA fragments synthesized by Invitrogen were used as site-directed mutagenesis primers, and their sequences were as follows:

847- Primer 1

SEQ ID NO: 128

CAAACTGTGCGGTCGTGAACTGGTTCGTGCGCAAA
847- Primer 2

SEQ ID NO: 129

TTTGCGCACGAACCAGTTCACGACCGCACAGTTTG

A vector comprising the DNA sequence of relaxin 800845 carrier was used as a template for site-directed PCR mutagenesis. The DNA Sequence of the 800847 precursor is as follows:

SEQ ID NO: 130

ATGAAGAAAAACATCGCGTTCCTGCTGAAACGTGACTCTTGGATGGAA
GAAGTTATCAAACTGTGCGGTCGTGAACTGGTTCGTGCGCAAATTGCGAT
CTGCGGTATGTCTACCTGGTCTAAACGTAAACCGACCGGTTACGGTTCTA
AACGTGACCTGTACTCTGCGCTGGCGAACAAATGCTGCCACGTTGGTTG
CACCAAACGTTCTCTGGCGCGTTTCTGCTAA

The amino acid sequence coded by the DNA sequence above is as follows:

SEQ ID NO: 25

MKKNIAFLLKRDSWMEEVIKLCGRELVRAQIAICGMSTWSKRKPTGYGSK
RDLYSALANKCCHVGCTKRSLARFC.

2. Inducible Expression of Small Amount of Recombinant Human Relaxin 800847

The procedure for the inducible expression of a small amount of recombinant human relaxin 800847 was the same as that described in Example 23.

The sequences of the resulting recombinant human relaxin analog 800847 chains were as follows:

SEQ ID NO: 134

chain B (WT) DSWMEEVIKLCGRELVRAQIAICGMSTWS

SEQ ID NO: 138

chain A (D^{A1}) DLYSALANKCCHVGCTKRSLARFC.

### Example 26

### Cloning and Expression of Recombinant Human Relaxin 800851

1. Construction of Recombinant Human Relaxin 800851 Expression Vector

The DNA sequence of relaxin 800851 was synthesized by site-directed PCR mutagenesis. Two single-stranded DNA fragments synthesized by Invitrogen were used as site-directed mutagenesis primers, and their sequences were as follows:

SEQ ID NO: 131

851-Primer 1 CGCGTTCCTGCTGAAACGTTGGATGGAAGAAGTTATC

SEQ ID NO: 132

851-Primer 2 GATAACTTCTTCCATCCAACGTTTCAGCAGGAACGCG

A vector comprising the DNA sequence of relaxin 800845 carrier was used as a template for site-directed PCR mutagenesis. The DNA Sequence of the 800851 precursor is as follows:

SEQ ID NO: 133

ATGAAGAAAAACATCGCGTTCCTGCTGAAACGTTGGATGGAAGAAGTT
ATCAAACTGTGCGGTCGTGACCTGGTTCGTGCGCAAATTGCGATCTGC
GGTATGTCTACCTGGTCTAAACGTAAACCGACCGGTTACGGTTCTAAA
CGTGACCTGTACTCTGCGCTGGCGAACAAATGCTGCCACGTTGGTTGC
ACCAAACGTTCTCTGGCGCGTTTCTGCTAA

The amino acid sequence coded by the DNA sequence above is as follows:

SEQ ID NO: 26

MKKNIAFLLKRWMEEVIKLCGRDLVRAQIAICGMSTWSKRKPTGYGSKRD
LYSALANKCCHVGCTKRSLARFC.

2. Inducible Expression of Small Amount of Recombinant Human Relaxin 800851

The procedure for the inducible expression of a small amount of recombinant human relaxin 800851 was the same as that described in Example 23.

The sequences of the resulting recombinant human relaxin analog 800851 chains were as follows:

SEQ ID NO: 140

chain B (Del^{B1B2},D^{B14}) WMEEVIKLCGRDLVRAQIAICGMSTWS

SEQ ID NO: 138

chain A (D^{A1}) DLYSALANKCCHVGCTKRSLARFC.

### Example 27

### Fermentation of Recombinant Human Relaxin 800828

1. Culturing of the Seed Strain

1) Strain: 800828 expression strain

2) Media:

a) LB medium: Yeast extract 5 g/L, typtone 10 g/L, NaCl 5 g/L, 121° C. 30 min, high temperature sterilized,

b) kanamycin stock solution: 50 mg/mL, 0.22 μm filter sterilized

c) Fermentation medium: Tryptone 20 g/L, Yeast extract 10 g/L, NaCl 10 g/L, Na₂HPO₄.12H₂O 4 g/L, KH₂PO₄ 2 g/L, K₂HPO4 2 g/L, MgSO₄ 1 g/L, glycerol 10 g/L, defoamer 5 mL, after dissolution, the mixture was loaded into 5 L of fermenter for sterilization at 121° C. for 30 min.

d) Supplementary medium: Tryptone 100 g/L, Yeast extract 50 g/L, glycerol 500 g/L, 121° C. sterilized for 30 min.

2. Fermentation Process Control

1) Seed Activation: A seed culture stored in a glycerol tube at −80° C. was thawed at room temperature, and 500 μL of the microbial suspension from the glycerol tube were inoculated into 50 mL of LB medium, followed by the addition of 5 μL of kanamycin stock solution, and culturing for 7 hours at 37° C., 200 rpm.

2) Fermenter uploading: 50 mL of activated seed culture was inoculated into 3 L of fermentation medium, followed by the addition of 30 mL of kanamycin. The conditions for the culture were as follows—temperature condition of the culture: 37° C., air flow rate: 0.5vvm, pressure of the fermenter: 0.04 to 0.05 mpa, DO: 30% and above, pH: about 7.0, maintained with ammonia.

3) About 4 hours after fermentation, samples were taken from the culture broth every hour to measure the OD600 value, and induction began at OD₆₀₀≧40.

4) Inducer: 4 mL of IPTG (1 mol/L, sterilized by passing through 0.22 μm filter membrane before use) was added, and the final concentration of IPTG was 1 mmol/L. The temperature was kept at 37° C. during the induction phase, and the other conditions remained unchanged. The fermentation broth was induced for 12 hours.

5) The fermenter was discharged when the induction ended, the OD600 of the culture broth was measured, and the sample was centrifuged at 5000 rpm for 20 min. The cells were collected, and stored at −80° C.

### Example 28

### Fermentation of Recombinant Human Relaxin 800814

1. Strain: 800828 expression strain

2. 5 L fermenter operation process:

1) Seed Strain Activation:

1 mL of the expression strain in glycerol was inoculated into 1000 mL of BMGY medium, and cultured for 20 hours at 30° C., 220 r/min (rpm).

2) Inoculation:

The activated seed culture was inoculated into 3 L of fermentation medium (H₃PO₄ 26.7 ml/L, CaSO₄ 0.93 g/L, K₂SO₄ 18.2 g/L, MgSO₄.7H₂O 14.9 g/L, KOH 4.13 g/L, glycerol 40 g/L), followed by the addition of 0.4% sterile PTM1 solution.

3) Initial Stage of Culture:

The expression strain entered into exponential growth phase after a period of adjustment (10 to 12 hr). A dissolved oxygen (DO) level of >30% required for cell growth was met by increasing the agitation speed and aeration rate. The rotation speed was increased by 50-100 rpm at a time. The fermentation temperature was controlled at 37° C., tank pressure: 0.04 to 0.05Mpa, pH: 7.0.

4) Glycerol-Supplemented Stage:

After the substrate in the initial medium was consumed (18 to 24 hr), 50% glycerol fed-batch was supplemented at a limited rate.

5) Methanol Induction Phase:

After 2 to 4 h of cell growth in the glycerol supplementation phase, glycerol supplementation was terminated, the cells were starved for 30 minutes to allow the glycerol to be completely exhausted, and then methanol was added for induction. After 70 to 96 hours, fermentation was stopped, the broth was centrifuged at 7000 rpm, and the supernatant was collected.

6) Yield of Protein Expression:

One ml of fermentation supernatant was analyzed for the protein content in the supernatant using SDS-PAGE, and appropriate expression was confirmed.

### Example 29

### Purification and Refolding of Recombinant Human Relaxin 800828

1. Purification of Inclusion Bodies

1) 100 g of wet weight E. coli was suspended in 300 ml of 50 mM Tris pH8.5 and 0.2% EDTA, and ultrasonicated. After centrifugation at 10000 rpm 4° C. for 1 hr, the supernatant was removed.

2) The pellet was suspended in 300 ml of 2M Urea, 50 mM Tris pH8.5, 0.2% EDTA. After centrifugation, the supernatant was removed, and this step was repeated once.

3) The pellet was suspended in 200 ml of 8M Urea, 50 mM Tris pH8.5, 0.2% EDTA. After centrifugation, the supernatant was removed.

4) The pellet was suspended in 200 ml of 50 mM Tris pH8.5, 0.2% EDTA. After centrifugation, the supernatant was removed and weighed.

5) The pellet was fully suspended in 6 volumes of 6M GdnCl 50 mM Tris pH8.5, 0.2% EDTA, 10 mM DTT, and resuspended completely by incubation at room temperature for 2 hr. After centrifugation at 10000 rpm 4° C. for 1 hr, the supernatant was collected.

6) Various intermediate wash samples were analyzed by electrophoresis.

2. Inclusion Bodies Refolding

1) Collected inclusion bodies were diluted in 2 volumes of 50 mM Tris pH8.5, 0.2% EDTA, fully mixed and placed at 4° C. overnight.

2) The refolding process was tracked using a reverse phase ultra performance liquid chromatography (RP-UPLC) test. After the reaction, the products were purified by preparative liquid phase, and the molecular weight was detected by liquid chromatography-mass spectrometry (LC-MS).

a) RP-UPLC Conditions: UPLC BioHClass Water, ACQUITY UPLC BEH 300 C4 columns (1.7 μm 2.1×100 mm), mobile phase A solution of 0.1% TFA and B solution of ACN, flow rate at 0.3 ml/min, and linear gradient of 0.5 min (10 B %) to 9 min (60 B %).

b) Preparation conditions: AutoPurifier Water, Kromasil 10-100-C18, 30×250 mm columns; mobile phase A solution of 0.1% TFA and B solution of ACN, flow rate at 40 ml/min, and linear gradient of 2 min (25% B) to 15 min (50% B).

c) LC-MS method: Finnigan LCQ Ad system, Jupiter C4 columns (5 u, 300 A, 250×4.6 mm), mobile phase A solution of 0.2% FA; B solution of 0.18% FA/CAN, and linear gradient of 0 min (5% B) to 20 min (50 B %).

3. Digestion conditions: 50 mM Tris pH8.5, 5 mM Ca2⁺, Trypsin 1:1300, CPB 1:50, substrate concentration of 1 mg/ml, reaction temperature of 30° C. The reaction process was detected by UPLC, the sample was then adjusted to pH 3-4 to terminate the reaction, and the sample was centrifuged to remove the pellet. The supernatant was filtered through a 0.22 μm membrane and purified by preparative RP-HPLC. The reverse phase preparative gradient was 2 min (20% B) to 15 min (45% B). Other test conditions were as mentioned above. The product (No. 828) was obtained by lyophilization, and its purity was analyzed by RP-UPLC and ion exchange chromatography HPLC (IEC-HPLC).

1) RP-UPLC conditions were as mentioned above.

2) IEC-HPLC conditions: UPLC BioHClass Water, Protein-Pak Hi Res CM columns (7 μm, 4.6×100 mm), A solution of 20 mM HEPES pH8.0, B solution of 0.5M NaCl 20 mM HEPES pH8.0, linear gradient of 1 min (0% B) to 10 min (100% B).

4. Cyclization conditions: The powder product was dissolved in 1 mM HCl and placed in an 80° C. water bath. The reaction progress was monitored by UPLC until completion. The test conditions were as mentioned above.

5. The prepared product, which served as the positive control i.e., sample 800828 (WT) with the original sequence, was dissolved in 20 mM NaAc pH5.0 for activity analysis.

The sequences of the resulting recombinant human relaxin 800828 chains (WT, wild-type) were as follows:

SEQ ID NO: 134

chain B (WT) DSWMEEVIKLCGRELVRAQIAICGMSTWS

SEQ ID NO: 135

chain A (WT) QLYSALANKCCHVGCTKRSLARFC.

### Example 30

### Purification and Identification of the Fermentation Product of Recombinant Human Relaxin 800814

A series of the molecules designed in the present disclosure contain amino acids with different charges at amino acid positions A₁ and B₁₄. Therefore, it is observed that the relaxin precursors expressed in yeast can be automatically cleaved within the cell, and the mature relaxin molecules are secreted into the supernatant of the culture medium. Intact relaxin molecules can thus be purified directly from the supernatant. The purification procedure was as follows:

Step 1: Supernatant Purified by Column

Broth supernatant was purified first using an AKTA purifier equipped with a Capto MMC (GE17-5318-03) column. The column was equilibrated with 20 mM NaAC pH4, the culture supernatant was adjusted to pH 4, and the sample was loaded. Eluted peaks were collected upon elution with 100 mM NaHCO₃ pH 11. The pH was adjusted to 3 for subsequent RP-HPLC purification. Conditions for reverse phase preparation purification were as follows: AutoPurifier Water, Kromasil 10-100-C18, 30×250 mm columns, mobile phase A solution of 0.1% TFA and B solution of ACN, flow rate of 40 ml/min, linear gradient of 2 min (20% B) to 15 min (50% B). Eluted peaks were collected. The obtained product of interest was lyophilized, and the resulting HPLC purity was 93%. The molecular weight was determined by LC-MS analysis to be 5953.0, which is consistent with the calculated molecular weight of 5952.9. The sequences of the resulting recombinant human relaxin analog 800814 chains are as follows:

SEQ ID NO: 137

chain B (D^{B14}) DSWMEEVIKLCGRDLVRAQIAICGMSTWS

SEQ ID NO: 138

chain A (D^{A1}) DLYSALANKCCHVGCTKRSLARFC.

Step 2: Identification of Disulfide Bonds

The above product was dissolved in 50 mM Tris pH8.0 and digested with trypsin (Sigma, Cat.T1426) at 37° C. overnight. The digestion reaction was terminated by the addition of 3 μl of 1M HCl. Fragments that were detectable by LC-MS are listed in Table 2. The presence of the disulfide bond between residues A24 and B23 was confirmed, and the other two disulfide bonds were present between residues A10, A11, A15 and B11.

TABLE 2
Disulfide bond identification result

| | | Calculated | Measured |
|---|---|---|---|
| | | molecular | molecular |
| Fragment No | Sequence | weight | weight |
| I | DSWMEEVIKLCGR | 3541.6 | 3541.7 |
| | DLYSALANKCCHVGCTKR | | |
| II | DLVR | 501.3 | 501.3 |
| III | SLAR | 445.5 | Not |
| | | | detected |
| IV | AQIAICGMSTWS | 1532.6 | 1532.6 |
| | FC | | |

### Example 31

### Peptide Fragment Analysis of Relaxin 800814

MS molecular weight and sequence coverage analysis of relaxin 800814 were analyzed using an Agilent 1260 LC-6530 Q-TOF-MS instrument. The mobile phase composition in liquid phase conditions were as follows:

Phase A: water (containing 0.1% formic acid); B phase: acetonitrile (containing 0.1% formic acid),

Column: Poroshell 300 SB-C8 2.1×75 mm 5 μm, column temperature: 50° C. The resulting elution gradients are shown in Table 3.

TABLE 3
Elution gradients

| | Intact molecule | | | DTT reduced sample | |
|---|---|---|---|---|---|
| | phase B | Flow rate | | phase B | Flow rate |
| Time | proportion % | mL/min | Time | proportion % | mL/min |
| 0 | 5 | 0.4 | 0 | 5 | 0.4 |
| 3 | 5 | 0.4 | 3 | 5 | 0.4 |
| 10 | 95 | 0.4 | 15 | 95 | 0.4 |
| 12 | 95 | 0.4 | 18 | 95 | 0.4 |
| 12.5 | 5 | 0.4 | 18.1 | 5 | 0.4 |
| 15 | 5 | 0.4 | 21 | 5 | 0.4 |

MS conditions were as follows: ion source: AJS ESI (+), ion source parameters: Nebulizer 40 psig, Drying Gas Temp 325° C., Gas Flow 10 l/min, Sheath Gas Temp 350° C., Sheath Gas Flow 12 l/min, Vcap 3500 V, Fragmentor 200 V, scan range: m/z 50-3200.

The molecular weight of relaxin 800814 was measured to be 5948.7996 Da, which is consistent with the theoretical value, thus confirming the correct expression.

Relaxin 800814 reduced peptide mapping was carried out by reducing the protein disulfides by DTT and performing sequence coverage analysis to confirm the correct protein expression. The conditions were as follows: 0.1 mg/mL of sample was incubated for 2 h with DTT (final concentration of 20 mM), then incubated for 30 min with IAA (final concentration of 40 mM) in darkness. The treated sample was analyzed by LC-MS. The results (Table 4) show that the sequence of the molecule completely matches the theoretical sequence, further confirming that the expected protein molecule was accurately expressed.

TABLE 4
Results of LC-MS detection analysis

| | | |
|---|---|---|
| relaxin | DSWMEEVIKLCGRDLVRAQIAICGMS | SEQ ID NO: 137 |
| 800814 | TWS | |
| chain B | | |
| relaxin | DLYSALANKCCHVGCTKRSLARFC | SEQ ID NO: 138 |
| 800814 | | |
| chain A | | |

### Example 31

### Preparation of the Human Relaxin Analog 800814PEG Derivative

1. 10.0 mg of the recombinant human relaxin 800814 sample (SEQ ID NO: 19, prepared in Example 19) was weighed in a 50 mL reaction tube and dissolved in 5.0 mL of 0.1M CH₃COOH/CH₃COONa buffer solution (pH4.5, 4° C.). The final concentration of recombinant human relaxin 800814 was 2.0 mg/mL (the preparation of 0.1 M glacial acetic acid/sodium acetate was as follows: 3.7 g of glacial acetic acid+3.2 g of sodium acetate+1000 mL of water).

2. 100 mg of aldehyde monomethoxy polyethylene glycol (m-PEG-CHO, 20 kDa) was weighed and dissolved in 2.0 mL of tetrahydrofuran/acetonitrile (1:1) mixed solvent, and the mixture was added to the above-mentioned recombinant human relaxin 800814 buffer solution (the ratio of PEG to recombinant human relaxin 800814 is 3:1).

3. 0.5 mg of sodium cyanoborohydride (NaCNBH₃) was weighed and dissolved in 1.0 mL of buffer solution (pH4.5) and added to the above-mentioned recombinant human relaxin 800814 buffer solution (the reductive agent was introduced at a ratio of 100:1 (reductive agent to modifying agent)).

4. The reaction mixture was maintained at room temperature (T=25° C.) for 1.0 and 2.0 h, and the reaction process was detected by RP-HPLC. When the reaction product was no longer generated, 1.0 mL of 10% glycine solution was added to the reaction sample which was then incubated for 10-30 min to quench the reaction.

5. Purification: the recombinant human relaxin 800814-PEG derivative (referred to as PEG-814) obtained from the reaction was separated and purified by an AKTA purifier 10 HPLC system equipped with an SP Sepharose Fast Flow cation exchange medium column (1.6 cm*18 cm). The equilibration buffer solution was 20 mmol/L HAC-NaAC, with pH=4.5. After a 10-fold dilution, the sample was loaded onto the column, the flow-through peak was collected, the column was washed with an elution buffer containing 1 mol/L NaCl, the eluted peaks were collected. The flow rate was 1.0 mL/min, and the detection wavelength was 280 nm. Human relaxin analog derivative PEG-814, a product of the present disclosure was obtained.

### Biological Evaluation

### Test Example 1

### In Vitro Bioactivity Assay of Human Relaxin and Analogs Thereof

The disclosed human relaxin analog (800814) binds to the receptor on THP-1 cells, and induces the production of cAMP in THP-1 cells. To determine the in vitro activity of human relaxin analog (800814), the generation of cAMP was detected. The wild-type human relaxin analog 800828 (WT) (prepared in Example 29) with the original sequence was used in this test as a positive control.

The cells used in the experiments, i.e., THP-1 (ATCC, Product Number: TIB-202™), were cultured in suspension at 37° C., 5% CO₂, and used for experiments or passage when the cells were preferably in the logarithmic growth phase. THP-1 cells can be passaged every 2-3 days, with a dilution ratio of 1:3 to 1:4. The medium was RPMI1640, with 0.05 mM b-mercaptoethanol and 10% FBS. On the day of the experiment, THP-1 cells in logarithmic growth phase were collected by centrifugation, and resuspended in DMEM/F12. The cell density was adjusted to 2×10⁶ cells/ml, and the cells were seeded in a 96-well plate (50 μl each well) and incubated in a 37° C. incubator for 30 minutes. The human relaxin analog was diluted 3-fold by diluent (DEME/F12+0.2% BSA+0.02 Polysorbate 80+2 μM forskolin+500 μM IBMX). 50 μl of the diluted human relaxin analog was added to the wells of the 96-well plate, and the samples were mixed for 2 minutes and incubated for 30 minutes in a 37° C. incubator. The cells were then centrifuged at 4100 rpm/min for 10 minutes (at 4° C.), 75 μl of the supernatant was removed, 50 μl of pre-cooled lysis buffer was added into each well (the whole operation was performed on ice), and samples were incubated on ice for 20 minutes, with shaking when necessary, and then the cells were centrifuged at 4100 rpm/min for 10 minutes (at 4° C.). The content of cAMP was detected with a CAMP ELISA KIT (CELL BIOLABS).

Data obtained in the above experiment was fit to a nonlinear curve using Graphpad Prism, and the EC₅₀ value (ng/ml) of CAMP induced in the THP-1 cells by human relaxin analog (800814) was determined. The result is shown in Table 5.

TABLE 5
in vitro activity assay of wild-type human relaxin
(800828) and human relaxin analog (800814)

| relaxin molecule | EC50 (ng/ml)* |
|---|---|
| 800828(WT) | 3.03 |
| 800814 | 1.50 |

*EC50 is the average of three experiments

These results indicate that, because of changes in molecular structure, the 800814 precursor molecule is able to undergo complete digestion within the host cells (eliminating the in vitro digestion step of expression product purification), and furthermore that the in vitro activity of the mature relaxin analog (800814), which is directly secreted into the supernatant, doubles when compared with the activity of the positive control molecule (800828 (WT)) (i.e., the EC50 decreased from 3.05 ng/ml to 1.50 ng/ml).

### Test Example 2

### In Vivo Biological Activity Detection of Human Relaxin and Analogs Thereof

ICR mice (female, purchased from SINO-BRITSH SIPPR/BK LAB. ANIMAL LTD., CO, Certificate No.: SCXK (Shanghai) 2008-0016, 18-20 g) were used in this test, and the maintenance environment was SPF grade. After their purchase, ICR mice were kept in the laboratory environment for two weeks in the following conditions: illumination: 12/12-hour light/dark cycle regulation, temperature: 20-25° C.; humidity: 40-60%. The wild-type human relaxin analog 800828 (WT, prepared in Example 29) with the original sequence was used as a positive control in this test.

One week before the experiment, each female ICR mouse was subcutaneously injected with 5 μg/100 μ1/animal of β-estradiol (water-insoluble, slightly soluble in oil), which was mixed with olive oil. Six days later, ICR mice with a body weight less than 22 g were excluded, and the rest of the animals were equally divided into groups according to body weight. The animals were subcutaneously injected with relaxin 800828 (WT) (6 m/100 μl/mouse), 800814 (6 μg/100 μl/mouse) or 0.1% benzopurpurine 4B saline (solvent control group). Relaxin 800828 (WT) and 800814 were dissolved in physiological saline containing 0.1% benzopurpurin 4B. 40 hours later, the pubic bones were removed, skin and muscle were removed from the bone, and the pubis width was measured under microscope. The average pubis width of the solvent control group was assigned to be 1, and the relative width was represented as the ratio of width of each administration group to that of solvent control group. The results are shown in Table 6.

TABLE 6
Pubic width

| Sample | relative pubic width |
|---|---|
| solvent control | 1 |
| Positive 800828(WT) | 1.24 |
| 800814 | 1.72* |

*The difference between 800814 and positive control molecule 800828(WT) is statistically significant.

These results indicate that, like 800814, the positive control molecule also promotes the growth of the pubic bone in mice, while 800814 molecule has significantly better efficacy than the positive control molecule (1.72 vs 1.24, p<0.05).

### Test Example 3

### In Vivo Half-Life Measurement of Human Relaxin and Analogs Thereof in Rats

A total of 12 experimental Sprague Dawley (SD) rats (6 male and 6 female) were used in this test (purchased from SINO-BRITSH SIPPR/BK LAB. ANIMAL LTD., CO, Certificate No.: SCXK (Shanghai) 2008-0016, 160-180 g), and the maintenance environment was SPF grade. SD rats were kept for 3 days in the laboratory environment, at a temperature of 20-25° C., with a humidity of 40-60%. The wild-type human relaxin analog 800828 (WT, prepared in Example 29) with the original sequence was used as a positive control in this test.

Rats were randomly divided into 3 groups, with 4 rats in each group, half males and half females. Control (saline) and human relaxin (analog 800814, WT 800828) were injected into the caudal vein of the rats (4 rats/sample) with a dose of 0.5 mg/kg/rat. Blood samples were taken from the rats' orbital sinuses at 0 h, 0.03 h, 0.08 h, 0.25 h, 0.5 h, 1 h, 1.5 h, 2 h, 4 h, 6 h and 8 h after the injections. Blood samples were centrifuged, and the supernatant was collected and stored at −20° C. for further analysis. After blood collection, the content of relaxin in the blood samples was detected using the Human relaxin-2 Quantikine ELISA Kit (R&D). The T_{1/2} of the test drug was calculated by T1/2 formula and EXCEL. The results are shown in Table 7.

TABLE 7
In vivo half-life measurement of human
relaxin and analogs thereof in rats

| Sample | T_{1/2} (hour) |
|---|---|
| 800828(WT) | 0.87 |
| 800814 | 0.88 |

The results show that the structural change of molecule 800814 according to the present disclosure does not affect its half-life.

### Test Example 4

### Long-Term In Vivo Activity Assay of the Human Relaxin Analog Derivative PEG-814

Experimental spontaneously hypertensive female rats (SHR) rats, 35 weeks old, weighing about 370 g, were available from Beijing Weitong Lihua Experimental Animal Technology Co. Ltd., cat number: 11400700009329. The maintenance environment was SPF grade. The test sample human relaxin analog derivative PEG-814 was prepared as in Example 31.

After purchase, 5 rats/cage, were kept in the laboratory environment for 1 week, with 12/12-hour light/dark cycle regulation, a temperature of 20-25° C., and humidity of 50-60%. Before the experiment, the basal blood pressure of the SHR rats was measured 2-3 times with a non-invasive blood pressure monitor (Softron, Item No: BP-98A). Rats with stable blood pressure that was not less than 170 mmHg were selected and randomly divided into the test drug group (PEG-814) and solvent control group (saline) (10 rats in each group). Relaxin PEG-814 or saline were injected into the caudal vein of the rats, 500 μl at a time (at 30 μg/day/rat), once daily (at 15:00 p.m.), for six consecutive weeks. Blood pressure was measured once a week, and body weight and blood pressure data were recorded.

The rats' body weight and blood pressure changes were calculated for each group using excel statistical software. Body weight and blood pressure data in the test group and the solvent group were subjected to t test, and blood pressure before and after administration in both the treatment group and the solvent group were compared for statistical significance and significant difference. The antihypertensive effect of PEG-814 was evaluated. The results are shown in Table 8.

TABLE 8
impact of PEG-814 on blood pressure and body weight of SHR rats

| | Index | | | |
|---|---|---|---|---|
| | Body Weight | | Blood pressure | |
| Group | PEG-814 | Solvent control | PEG-814 | Solvent control |
| Before | 362 ± 16.54 | 355 ± 18.97 | 203 ± 7.16 | 203 ± 10.27 |
| admini- | | | | |
| stration | | | | |
| 1 week | 363 ± 15.79 | 355 ± 18.12 | 193 ± 9.78# | 198 ± 14.12 |
| 2 weeks | 360 ± 18.22 | 353 ± 18.43 | 192 ± 24.06 | 208 ± 13.56 |
| 3 weeks | 360 ± 16.77 | 346 ± 17.25 | 199 ± 14.09* | 214 ± 18.06 |
| 4 weeks | 363 ± 17.62 | 351 ± 19.87 | 197 ± 17.06 | 207 ± 15.74 |
| 5 weeks | 361 ± 20.32 | 353 ± 21.64 | 199 ± 15.8* | 214 ± 13.96 |
| 6 weeks | 362 ± 17.41 | 357 ± 20.85 | 197 ± 15.71 | 209 ± 9.1 |

Note:
*p < 0.05, vs solvent control group;
#p < 0.05, vs before administration

The results shows that PEG-814 exhibits significant efficacy (reducing blood pressure) and has a long-lasting effect when administrated once daily; and PEG-814 has no effect on body weight. Conventional relaxin (non-PEG-modified) requires continuous intravenous administration.

1. A human relaxin analog, comprising chain A and chain B, the amino acid sequences of chain A and chain B being represented by the following formulas:
chain A: A₁LYSALANKCCHVGCTKRSLARFC,
and
chain B: DSWMEEVIKLCGRB₁₄LVRAQIAICGMSTWS;
wherein
A₁ is selected from the group consisting of Q, D, E, and W;
B₁₄ is selected from the group consisting of E, D and N;
the D residue at B₁ and the S residue at B₂ are optionally absent simultaneously; and
when A₁ is Q, B₁₄ is neither E nor D.

2. The human relaxin analog according to claim 1, the amino acid sequences of chain A and chain B being represented by the following formulas:
chain A: A₁LYSALANKCCHVGCTKRSLARFC,
and
chain B: DSWMEEVIKLCGRB₁₄LVRAQIAICGMSTWS;
wherein
A₁ is selected from the group consisting of D, E and W;
B₁₄ is selected from the group consisting of E, D and N; and
when A₁ is Q, B₁₄ is neither E nor D.

3. The human relaxin analog according to claim 1, wherein B₁₄ is D.

4. The human relaxin analog according to claim 1, wherein the amino acid sequences of chain A and chain B are selected from the group consisting of:
SEQ ID NO: 137
chain B: DSWMEEVIKLCGRDLVRAQIAICGMSTWS,
SEQ ID NO: 138
chain A: DLYSALANKCCHVGCTKRSLARFC;
SEQ ID NO: 137
chain B: DSWMEEVIKLCGRDLVRAQIAICGMSTWS,
SEQ ID NO: 135
chain A: QLYSALANKCCHVGCTKRSLARFC;
SEQ ID NO: 137
chain B: DSWMEEVIKLCGRDLVRAQIAICGMSTWS,
SEQ ID NO: 136
chain A: ELYSALANKCCHVGCTKRSLARFC;
SEQ ID NO: 139
chain B: DSWMEEVIKLCGRNLVRAQIAICGMSTWS,
SEQ ID NO: 135
chain A: QLYSALANKCCHVGCTKRSLARFC;
SEQ ID NO: 140
chain B: WMEEVIKLCGRDLVRAQIAICGMSTWS,
SEQ ID NO: 138
chain A: DLYSALANKCCHVGCTKRSLARFC;
SEQ ID NO: 134
chain B: DSWMEEVIKLCGRELVRAQIAICGMSTWS,
SEQ ID NO: 141
chain A: WLYSALANKCCHVGCTKRSLARFC;
and
SEQ ID NO: 134
chain B: DSWMEEVIKLCGRELVRAQIAICGMSTWS,
SEQ ID NO: 138
chain A: DLYSALANKCCHVGCTKRSLARFC.

5. The human relaxin analog according to claim 1, wherein the chain B is linked to the chain A by a linker sequence, and the linker sequence has a length of 1 to 15 amino acid residues.

6. The human relaxin analog according to claim 5, wherein the linker sequence has an amino acid sequence selected from the group consisting of:
L1: KR,
SEQ ID NO: 27
L2: KRKPTGYGSRKKR,,
SEQ ID NO: 28
L3: KRKPTGYGSRKR,,
SEQ ID NO: 29
L4: KRGGGPRR,,
SEQ ID NO: 30
L5: KRGGGPKR,,
SEQ ID NO: 31
L6: KRKPTGYGSKR,,
and
SEQ ID NO: 32
L7: KRSLKR.

7. The human relaxin analog according to claim 1, wherein the N terminus of the human relaxin analog is linked to a signal peptide sequence, and the signal peptide sequence has a length of 4 to 15 amino acid residues.

8. The human relaxin analog according to claim 7, wherein the signal peptide sequence is selected from the group consisting of:
SEQ ID NO: 33
S1: EEGEPK,,
SEQ ID NO: 34
S2: EEGEPKR,,
and
SEQ ID NO: 35
S3: MKKNIAFLLKR,.

9. A human relaxin analog derivative, comprising a human relaxin analog according to claim 1 modified by a PEG, wherein the PEG has a molecular weight of 5 to 100 KDa and the PEG molecule is a branched-chain PEG or a linear-chain PEG.

10. An expression precursor for the human relaxin analog according to claim 1, comprising the amino acid sequence of one or more selected from the group consisting of SEQ ID NO: 1 to SEQ ID NO: 26.

11. A polynucleotide encoding the expression precursor according to claim 10.

12. An expression vector comprising the polynucleotide according to claim 11.

13. A host cell transformed with the expression vector according to claim 12.

14. The host cell according to claim 13, wherein the host cell is a bacterial cell.

15. The host cell according to claim 13, wherein the host cell is a yeast cell.

16. A pharmaceutical composition comprising:
at least one of a human relaxin analog according to claim 1, and a human relaxin analog derivative comprising the human relaxin modified by a branched-chain or linear-chain PEG having a molecular weight of 5 to 100 KDa; and
one or more pharmaceutically acceptable carrier(s), diluent(s) or excipient(s).

17. An injectable solution comprising a soluble form of the pharmaceutical composition according to claim 16.

18. (canceled)

19. A method for treating or preventing a fibrotic or cardiovascular disease in a subject in need thereof, comprising administering to the subject the pharmaceutical composition according to claim 16.

20. A method for treating or preventing a fibrotic or cardiovascular disease in a subject in need thereof, comprising administering to the subject a pharmaceutical composition comprising
at least one of a human relaxin analog according to claim 4, and a human relaxin analog derivative comprising the human relaxin modified by a branched-chain or linear-chain PEG having a molecular weight of 5 to 100 KDa; and
one or more pharmaceutically acceptable carrier(s), diluent(s) or excipient(s).

21. A method for treating or preventing a fibrotic or cardiovascular disease in a subject in need thereof, comprising administering to the subject a pharmaceutical composition comprising a human relaxin analog having the amino acid sequences of:
SEQ ID NO: 137
chain B: DSWMEEVIKLCGRDLVRAQIAICGMSTWS,
and
SEQ ID NO: 138
chain A: DLYSALANKCCHVGCTKRSLARFC;
wherein the chain A and chain B are covalently linked, or
a human relaxin analog derivative comprising the human relaxin modified by a branched-chain or linear-chain PEG having a molecular weight of 5 to 100 KDa; and
one or more pharmaceutically acceptable carrier(s), diluent(s) or excipient(s).
